(19) United States
(12) Patent Application Publication
(10) Pub. No.: US 2026/0028425 A1
(43) Pub. Date: Jan. 29, 2026

(54) ANTI-BSSL ANTIBODIES FOR THE TREATMENT OF CANCER

(71) Applicant: LIPUM AB, Umeå (SE)

(72) Inventors: Susanne LINDQUIST, Röbäck (SE); Lennart LUNDBERG, Billdal (SE); Olle HERNELL, Umeå (SE)

(21) Appl. No.: 19/120,485

(22) PCT Filed: Oct. 13, 2023
(86) PCT No.: PCT/SE2023/051022

§ 371 Date: Apr. 11, 2025

(30) Foreign Application Priority Data
Oct. 14, 2022 (SE) 2251206-5

(51) Int. Cl. C07K 16/40; A61K 39/00; A61P 35/00

(52) U.S. Cl. CPC C07K 16/40; A61P 35/00; A61K 2039/505; C07K 2317/34; C07K 2317/565

(57) ABSTRACT

The invention relates to antibody, or an antigen-binding fragment thereof, binding specifically to bile salt-stimulated lipase (BSSL) for use in treatment of a BSSL-expressing cancer in a subject. The antibody, or the antigen-binding fragment thereof, binds to an epitope present in an N-terminal part of BSSL ranging from a N-terminus of BSSL up to amino acid residue 300 in BSSL but an active site of BSSL.

### TECHNICAL FIELD

The invention relates to cancer treatment, and in particular to the use of anti-BBSL antibodies in such cancer treatment.

### BACKGROUND

Bile Salt-Stimulated Lipase (BSSL), also known as Bile Salt-Dependent Lipase (BSDL), Carboxyl Ester Lipase (CEL) or Bile Salt-Activated Lipase (BAL), is a lipolytic enzyme encoded by the CEL gene. BSSL is expressed in the exocrine pancreas and is secreted into the intestinal lumen in all species so far investigated and aids in the digestion of lipids.

In some species, including humans, primates, dogs, cats and mice, BSSL is also expressed in lactating mammary gland and secreted in the milk. Moreover, BSSL has been found in low, but significant levels in serum of healthy individuals and to be involved in lipoprotein metabolism and modulation of atherosclerosis. BSSL has also been found to have a role in inflammatory processes.

Cancer Genetics 2019, 239:54-61 investigated the link between BSSL expression of breast cancer tissues and the survival of breast cancer patients by analyzing the Cancer Genome Atlas Breast Carcinoma (TCGA-BFCA) level 3 data. Analysis of 1104 cases of tumor data showed that BSSL was over-expressed in breast cancer. There was a relationship between high BSSL expression and clinicopathologic features. The high BSSL expression group had a lower survival. By analyzing the area under the receiver operating characteristic ROC curve (AUC) of BSSL, it was found to have a limited diagnostic capability.

U.S. Pat. Nos. 7,557,193 and 8,367,062 disclose a glycopeptide comprising between 1 and 40 repeated C-terminal polypeptides with 11 amino acids (J28 peptide) of BSDL or human fetoacinar pancreatic protein (FAPP). The repeated C-terminal polypeptides are glycosylated and bear glycosylated epitopes giving rise to a specific immunological reaction with induced antibodies in a patient suffering from type 1 diabetes.

U.S. Publication No. 2010/0124555 discloses a method for producing an antigen-binding compound suitable for use in the treatment of cancer, the antigen-binding compounds and their uses.

There is still a need for a treatment of cancer, and in particular of BSSL-expressing cancers.

### SUMMARY

It is a general objective to provide a treatment of BSSL-expressing cancers.

This and other objectives are met by the embodiments as disclosed herein.

The present invention is defined in the independent claim. Further embodiments of the invention are defined in the dependent claims.

An aspect of the invention relates to an antibody, or an antigen-binding fragment thereof, binding specifically to BSSL for use in treatment of a BSSL-expressing cancer in a subject. The antibody, or the antigen-binding fragment thereof, binds to an epitope present in an N-terminal part of BSSL ranging from a N-terminus of BSSL up to amino acid residue 300 in BSSL but excludes an active site of BSSL.

The present invention relates to antibodies, or antigen-binding fragments thereof, binding to the N-terminal part of BSSL. Such antibodies, or antigen-binding fragments thereof, are cable of reducing the primary tumor size of BSSL-expressing cancers and may additionally have an effect in terms of inhibiting metastases from such primary tumors. A significant advantage of the antibodies, or antigen-binding fragments thereof, is that they do not bind to the active site of BSSL and thereby do not interfere with the enzymatic activity of BSSL. Hence, any side effects otherwise associated with inhibited BSSL activity are thereby avoided or at least reduced.

### BRIEF DESCRIPTION OF THE DRAWINGS

The embodiments, together with further objects and advantages thereof, may best be understood by making reference to the following description taken together with the accompanying drawings, in which:

FIG. 1 illustrates normalization of primary tumor growth data and normalization of metastasis of all cell lines treated with SOL-116 relative to human IgG4 S241P isotype control. Data shown as mean±SEM, two-tailed Student t-test **p<0.01.

FIG. 2 illustrates effect of SOL-116 on LXFS-1129 primary tumor size and metastasis. Data shown as mean±SEM, two-tailed Student t-test *p<0.05 and **p<0.01.

FIG. 3 illustrates effect of SOL-116 on CXF-1788 primary tumor size and metastasis. Data shown as mean±SEM, two-tailed Student t-test *p<0.05.

FIG. 4 illustrates effect of SOL-116 on CXF-158 primary tumor size and metastasis. Data shown as mean±SEM, two-tailed Student t-test *p<0.05.

FIG. 5 illustrates effect of SOL-116 on LXFS-2156 primary tumor size and metastasis. Data shown as mean±SEM, two-tailed Student t-test *p<0.05.

FIG. 6 illustrates effect of SOL-116 on CXF-2836 primary tumor size and metastasis. Data shown as mean±SEM, two-tailed Student t-test **p<0.01.

FIG. 7 illustrates effect of SOL-116 on CXF-533 primary tumor size and metastasis. Data shown as mean±SEM.

FIG. 8 illustrates effect of anti-BSSL antibodies on LXFS-1129 primary tumor size and metastasis. Data shown as mean±SEM. One-way ANOVA (p=0.0009) analysis followed by a two-tailed Student t-test *p <0.05, **p<0.01, and ***p<0.001.

FIG. 9 illustrates effect of anti-BSSL antibodies on LXFS-1129 primary tumor size. Negative control and SOL-116 data from Example 2 were combined with negative control and SOL-116 data obtained in Example 3. Data shown as mean±SEM. One-way ANOVA (p=0.0003) followed by two-tailed Student's t test *p<0.05, **p<0.01, ***p<0.001, and ****p<0.0001.

FIG. 10 illustrates effect of anti-BSSL antibodies on CXF-158 primary tumor size and metastasis. Data shown as mean±SEM. One-way ANOVA (p=0.0259) analysis followed by a two-tailed Student's t-test, *p<0.05.

FIG. 11 illustrates effect of anti-BSSL antibodies on CXF-158 primary tumor size. Negative control and SOL-116 data from Example 2 were combined with negative control and SOL-116 data obtained in Example 3. Data shown as mean±SEM. One-way ANOVA (p=0.0013) followed by two-tailed Student's t test *p<0.05, **p<0.01, and ***p<0.001.

FIG. 12 illustrates effect of anti-BSSL antibodies on LXFS-2156 primary tumor size and metastasis. Data shown as mean±SEM. One-way ANOVA (p=0.0088) analysis followed by a two-tailed Student's t-test, *p<0.05 and **p<0.01.

FIG. 13 illustrates effect of anti-BSSL antibodies on LXFS-2156 primary tumor size. Negative control and SOL-116 data from Example 2 were combined with negative control and SOL-116 data obtained in Example 3. Data shown as mean±SEM.

FIG. 14 illustrates effect of anti-BSSL antibodies on CXF-533 primary tumor size and metastasis. Data shown as mean±SEM.

FIG. 15 illustrates effect of anti-BSSL antibodies on CXF-533 primary tumor size. Negative control and SOL-116 data from Example 2 were combined with negative control and SOL-116 data obtained in Example 3. Data shown as mean±SEM.

### DETAILED DESCRIPTION

The invention relates to cancer treatment, and in particular to the use of anti-BBSL antibodies in such cancer treatment.

### Definitions

The term “isolated” when used in connection with antibodies, or antigen-binding fragments thereof, means that the antibody, or the antigen-binding fragment thereof, has been removed from its original environment. An isolated antibody, or an isolated antigen-binding fragment thereof, is intended to refer to an antibody, or an antigen-binding fragment thereof, that is substantially free of other antibodies, or antigen-binding fragments thereof, having different antigenic specificities. Hence, an isolated antibody, or antigen-binding fragment thereof, that specifically binds BSSL, in particular human BSSL (hBSSL), is substantially free of antibodies, or antigen-binding fragments thereof, that specifically bind antigens other than BSSL, in particular hBSSL. An isolated antibody, or antigen-binding fragment thereof, that specifically binds hBSSL may, however, have cross-reactivity to other antigens, such as BSSL molecules from other species, such as murine BSSL (mBSSL) from mouse. Moreover, an isolated antibody, or antigen-binding fragment thereof, may be substantially free of other cellular material and/or chemicals. For example, the isolated antibody, or an antigen-binding fragment thereof, may be purified to greater than 95% or 99% purity as determined by, for example, electrophoretic, e.g., sodium dodecyl sulfate-polyacrylamide gel electrophoresis (SDS-PAGE), isoelectric focusing (IEF), capillary electrophoresis, or chromatographic, e.g., ion exchange or affinity chromatography. It is appreciated that isolated antibodies, or antigen-binding fragments thereof, are referred to herein even though the word “isolated” is not explicitly mentioned each time the term “antibodies, or antigen-binding fragments thereof” are used.

The term “antigen-binding fragment” means a fragment or part of an antibody, which substantially retains antigen-binding properties. An antigen-binding fragment is a portion or region of an antibody molecule that retains all or a significant part of the antigen binding of the corresponding full-length antibody. An antigen-binding fragment may comprise one or more complementarity-determining region (CDR) sequences of the antibody or part of these CDR sequences, part or all of the heavy chain variable region (HCVR), part or all of the light chain variable region (LCVR), or a combination thereof. In an embodiment, an antigen-binding fragment of an antibody may be composed of a consecutive amino acid sequence of the antibody, from which it is obtained, or may be composed of different parts of the amino acid sequence of the antibody, joined together with or without linker(s). Examples of antigen-binding fragments are single-chain variable fragments (scFv), Fab fragments, F(ab′)₂ fragments, F(ab′)₃ fragments, Fab′ fragments, Fv fragments, dAb fragments, isolated complementarity determining regions (CDRs) and nanobodies.

A “single-chain variable fragment” (“scFv”) is a fusion protein of the variable regions of the heavy and light chains of immunoglobulins, connected with a short linker peptide of typically about 10 to 25 amino acids. scFvs of the same or different type (having an affinity for the same or different epitope(s)) may be combined in different ways as is known to the person skilled in the art. Non-limiting examples of such combinations are tandem di-scFv, diabodies, tandem tri-scFv or tri (a) bodies.

The term “epitope” refers to the part of an antigen that is recognized by the immune system, such as by antibodies, or antigen-binding fragments thereof. Epitope is also referred to as antigenic determinant.

As used herein, the terms “that binds to”, “having affinity for”, “affinity” and the like refer to the property of an antibody, or an antigen-binding fragment thereof, of binding to a target molecule. Standard assays to evaluate the binding ability of an antibody or an antigen-binding fragment towards a target molecule include for example, enzyme immunoassays (EIA), such as enzyme-linked immunosorbent assay (ELISA), Western blot, radioimmunoassay (RIA), surface plasmon resonance (SPR), LUMINEX® Multiplex Assay and flow cytometry analysis.

By “specifically binds to”, “specifically binding to” and the like it is meant that the antibody, or the antigen-binding fragment thereof, selectively binds to the target antigen while not showing any biologically relevant binding to other molecules. The specificity of an antibody, or an antigen-binding fragment thereof, can be determined based on affinity and/or avidity. The affinity, represented by the equilibrium constant for the dissociation of an antigen with the antibody, or the antigen-binding fragment thereof, (K_{D}) is a measure of the binding strength between an antigenic determinant, i.e., epitope, and an antigen-binding site on the antibody, or the antigen-binding fragment thereof. The lower the value of K_{D}, the stronger the binding strength between the antigenic determinant and the antibody, or the antigen-binding fragment thereof. Alternatively, the affinity can also be expressed as the affinity constant (K_{A}), which is 1/K_{D}. As will be clear to the skilled person, affinity can be determined in a manner known per se, depending on the specific antigen of interest. Typically, antibodies, or antigen-binding fragments thereof, will bind to their antigen with an equilibrium dissociation constant (K_{D}) of 10⁻⁵ to 10⁻¹² moles/liter (M) or less, and preferably 10⁻⁷ to 10⁻¹² M or less and more preferably 10⁻⁸ to 10⁻¹² M, i.e., with an affinity constant (K_{A}) of 10⁵ to 10¹² M⁻¹ or more, and preferably 10⁷ to 10¹² M⁻¹ or more and more preferably 10⁸ to 10¹² M⁻¹. Generally, any K_{D} value greater than 10⁻⁴ M (or any K_{A} value lower than 10⁴ M⁻¹) is considered to indicate non-selective binding, i.e., lack of specificity for the antigen. Preferably, an antibody, or an antigen-binding fragment thereof, of the embodiments will bind to a BSSL with a dissociation constant of less than 500 nM, preferably less than 200 nM, more preferably less than 10 nM, such as less than 5 nM.

Herein, all amino acids in the variable regions, including the herein described CDRs, are consequently numbered according to the IMGT unique numbering as defined by Marie-Paule Lefranc (Developmental and Comparative Immunology (2003) 27(1): 55-77).

It is known that certain cancer types express BSSL, including breast cancer and pancreatic cancer. In particular, BSSL has then mainly been used as a biomarker for patient stratification and prognosis. In particular, high BSSL expression was associated with low survival in breast cancer patients.

U.S. Pat. Nos. 7,557,193 and 8,367,062 investigated the effect of the monoclonal IgM antibody 16D10 (mAb 16D10) in the treatment of exocrine pancreatic cancer. mAb 16D10 is directed against the glycosylated epitopes expressed specifically on the C-terminal domain of FAPP and BSDL. mAb 16D10 resulted in lower tumor growth rate and lower median tumor volume as compared to control. U.S. Publication No. 2010/0124555 compared mAb 16D10 with a mouse monoclonal antibody mAb J28 specific to the fucosylated J28 glycopeptide derived from O-glycosylation of the oncofetal form of FAPP and an anti-BSDL polyclonal antibody pAbL64 in a pancreatic tumor model. mAb J28 is said to bind to a different epitope on BDSL and/or FAPP as compared to mAb 16D10. mAb 16D10 but not mAb J28 nor pAbL64 could reduce pancreatic tumor cell growth although all of the antibodies were directed against BDSL and/or FAPP. The conclusion was that the ability of a monoclonal antibody to stimulate apoptotic cellular death of pancreas cancer cells was dependent on the localization of the antigen of mAb 16D10 in membrane lipid raft microdomains.

The present invention is based on the finding that specific anti-BSSL antibodies, or antigen-binding fragments thereof, targeting the N-terminal part of BSSL are useful in treatment of BSSL-expressing cancer. These anti-BSSL antibodies, however, had no significant effect on cancer types expressing no or low levels of BSSL. This means that BSSL is useful not merely as a biomarker for predicting patient survival but is also a target for treatment of BSSL-expressing cancer. These effects of the anti-BSSL antibodies were highly surprising given that BSSL has no known effect on cancer cells but is rather a lipolytic enzyme (EC 3.1.1) that hydrolyzes dietary fat, cholesteryl esters and fat-soluble vitamins in the duodenum.

The antibodies, or antigen-binding fragments thereof, of the invention bind to different epitope(s) on BSSL as compared to the monoclonal antibody mAb 16D10 disclosed as being effective in inducing apoptosis in exocrine pancreatic cancer cells. This monoclonal antibody mAb 16D10 is said to bind to the glycosylated C-terminal part of BSDL/FAPP. Binding of mAb 16D10 to this glycosylated C-terminal part of BSDL/FAPP causes an induction of apoptosis of the exocrine pancreatic cancer cells through activation of caspase-3, caspase-8 and caspase-9, and through poly-ADP ribose polymerase (PARP) cleavage. This means that the cancer cells must, for mAb 16D10 to be effective, not only express BSDL/FAPP but also caspase-3, caspase-8 and/or caspase-9, and PARP.

The antibodies, or antigen-binding fragments thereof, of the invention bind specifically to an epitope present in an N-terminal part of BSSL. This N-terminal part of BSSL ranges from a N-terminus of BSSL up to amino acid residue 300 in BSSL but excludes an active site of BSSL.

Hence, an aspect of the invention relates to an antibody, or an antigen-binding fragment thereof, binding specifically to BSSL for use in treatment of a BSSL-expressing cancer in a subject. The antibody, or the antigen-binding fragment thereof, binds to an epitope present in an N-terminal part of BSSL ranging from a N-terminus of BSSL up to amino acid residue in BSSL but excludes an active site of BSSL.

Human BSSL consists of 722 amino acid residues (SEQ ID NO: 46) with a C-terminal part containing 16 proline-rich repeats of 11 residues with O-linked carbohydrate and is highly glycosylated. The catalytic triad in the active site of BSSL consists of serine at position 194 (Ser 194), aspartic acid at position 320 (Asp 320) and histidine at position 435 (His 435) (Protein Science (1997) 6:73-79; Journal of Molecular Biology (2001) 312 (3): 511-523). Human BSSL as expressed initially contains a 20 amino acid signal peptide that is cleaved off in connection with secretion of the protein. Reference to amino acid positions as used herein relate to amino acid positions in mature human BSSL protein following cleavage of the signal peptide, i.e., SEQ ID NO: 46. Human BSSL contains up to 17 proline-rich repeats in the C-terminal part resulting in a length of 733 amino acid residues (UniProt accession number P19835). However, herein reference to human BSSL and to amino acid residues in human BSSL is to the amino acid sequence of SEQ ID NO: 46. The antibodies, or the antigen-binding fragments thereof, as disclosed herein, though, bind specifically also to other forms of human BSSL.

Hence, in an embodiment, the antibody, or the antigen-binding fragment thereof, binds to an epitope comprising amino acids within the N-terminal part of BSSL from the N-terminus up to including amino acid residue 300 in SEQ ID NO: 46 but excludes amino acid residue 194 (and amino acid residues 320 and 435) in SEQ ID NO: 46.

The antibody, or the antigen-binding fragment thereof, of the invention therefore binds outside of the active site of BSSL. Accordingly, in an embodiment, the antibody, or the antigen-binding fragment thereof, does not affect an enzymatic lipase activity of BSSL. As used herein, “does not affect an enzymatic lipase activity” of BSSL means that that the lipase activity of a BSSL enzyme, to which an antibody, or an antigen-binding fragment thereof, of the invention is bound, is at least 80% of the lipase activity of the BSSL enzyme without any bound antibody, or antigen-binding fragment thereof. In particular, the lipase activity of the BSSL, to which the antibody, or the antigen-binding fragment thereof, of the invention is bound, is preferably at least 85%, and more preferably at least 90%, such as at least 95%, or even higher, such as at least 96%, at least 97%, at least 98%, or at least 99% of the lipase activity of the BSSL enzyme without any bound antibody, or antigen-binding fragment thereof.

The epitope, to which the antibody, or the antigen-binding fragment thereof, of the invention binds is located in the N-terminal part of BSSL and thereby not in the active site of BSSL. This has the advantage that the antibodies, or antigen-binding fragments thereof, are less likely to cause negative side effects as they do not significantly affect the enzymatic lipase activity of BSSL.

The BSSL structure has been described as a having a large core region consisting of a twisted, 11-stranded beta-sheet surrounded by alpha helices and connecting loops. At the N-terminus there is a smaller 3-stranded beta-sheet. The structure has been likened to a left-handed oven-glove with the palm containing the active site triad close to the “thumb”. With this likeness, the small N-terminal beta-sheet is located on the back of the hand close to the “little finger”. The part of the BSSL structure, which interacts with the antibody, or the antigen-binding fragment thereof, is located at the small N-terminal beta-sheet and the C-terminal part of alpha C, the third alpha helix in the structure. In other words, this region bound by an antibody, or an antigen-binding fragment thereof, is not close to the active site of BBSL but on the opposite side of BSSL.

In an embodiment, the antibody, or the antigen-binding fragment thereof, binds to an epitope of BSSL comprising amino acid residues 7-12 (strand 1 and 2 of the beta-sheet, SEQ ID NO: 1) and 42-55 (loop region leading into strand 3 of the beta-sheet, SEQ ID NO: 2). The epitope is rather flat with only a few characteristic residues sticking out, namely Tyr7, Phe12 and Gln52. The loop region of 47-55 is well defined and forms a uniform surface. Proline 47 is important for a stacking interaction with Tyr31 of an antibody, or an antigen-binding fragment thereof, but as a whole the surface is flat here. Hence, in an embodiment, the antibody, or the antigen-binding fragment thereof, binds to an epitope of BSSL comprising a first surface comprising, or defined by, an amino acid sequence according to SEQ ID NO: 1 and a second surface comprising, or defined by, an amino acid sequence according to SEQ ID NO: 2. In a particular embodiment, the first surface comprises, or is defined by, an amino acid sequence according to SEQ ID NO: 3 corresponding to amino acid residues 1-12 of hBSSL.

In an embodiment, the antibody, or the antigen-binding fragment thereof, may further specifically bind to another surface, i.e., a third surface of BSSL, such as hBSSL. In an embodiment, this third surface comprises, or is defined by, an amino acid sequence according to SEQ ID NO: 4, corresponding to amino acid residues 84-101 of hBSSL, an amino acid sequence according to SEQ ID NO: 5, corresponding to amino acid residues 174-180 of hBSSL, or an amino acid sequence according to SEQ ID NO: 6, corresponding to amino acid residues 283-294 of hBSSL.

In an embodiment, the antibody, or antigen-binding fragment thereof, may specifically bind to a first peptide comprising, such as consisting of, SEQ ID NO: 1, a second peptide comprising, such as consisting of, SEQ ID NO: 2 and a third peptide comprising, such as consisting of, SEQ ID NO: 4. Alternatively, instead of binding to the shorter amino acid sequence according to SEQ ID NO: 1, such an antibody, or antigen-binding fragment thereof, may bind to the longer amino acid sequence according to SEQ ID NO: 3.

In another embodiment, the antibody, or antigen-binding fragment thereof, binds specifically to a first peptide comprising, such as consisting of, SEQ ID NO: 1, a second peptide comprising, such as consisting of SEQ ID NO: 2, and a third peptide comprising, such as consisting of, SEQ ID NO: 5. Alternatively, instead of binding to the shorter amino acid sequence according to SEQ ID NO: 1, such an antibody, or antigen-binding fragment thereof, may specifically bind to the longer amino acid sequence according to SEQ ID NO: 3.

In a further embodiment, the isolated antibody, or antigen-binding fragment thereof, specifically binds to a first peptide comprising, such as consisting of SEQ ID NO: 1, a second peptide comprising, such as consisting of SEQ ID NO: 2, and a third peptide comprising, such as consisting of, SEQ ID NO: 6. Alternatively, instead of binding to the shorter amino acid sequence according to SEQ ID NO: 1, such an antibody, or antigen-binding fragment thereof, may bind to the longer amino acid sequence according to SEQ ID NO: 3.

In an embodiment, the antibody is a polyclonal antibody. The term “polyclonal antibody” as used herein refers to a collection of antibodies that react against a specific antigen, but in which collection there may be different antibody molecules, for example, identifying different epitopes on the antigen. Polyclonal antibodies are typically produced by inoculation of a suitable mammal and are purified from the mammal's serum.

In a preferred embodiment, the antibody is a monoclonal antibody. The term “monoclonal antibody” as used herein refer to an antibody having monovalent affinity, meaning that each antibody molecule in a sample of the monoclonal antibody binds to the same epitope on the antigen. Monoclonal antibodies may be made by identical immune cells that are clones of a unique parent cell, for example a hybridoma cell line, and may also be constructed by genetic engineering, including phage display selection from human immunoglobulin libraries.

A full-length antibody comprises two heavy chains and two light chains inter-connected by disulfide bonds. Each heavy chain contains a heavy chain variable region (HCVR) and first, second and third constant regions (CH1, CH2 and CH3). In this disclosure the terms V_{H}, VH and HCVR are used interchangeably. Each light chain contains a light chain variable region (LCVR), and a light chain constant region (CL). In this disclosure the terms V_{L}, VL and LCVR are used interchangeably.

The HCVR and LCVR regions can be further subdivided into regions of hypervariability, termed complementarity-determining regions (CDR), interspersed with regions that are more conserved, termed framework regions (FR or FW). Each HCVR and LCVR is composed of three CDRs and four FRs/FWs, arranged from N-terminus to C-terminus in the following order: FW1, CDR1, FW2, CDR2, FW3, CDR3, FW4.

It is well known in the art that a paratope, also known as the antigen-binding site, is the part of an antibody, or antigen-binding fragment thereof, which recognizes and binds to a surface of an antigen denoted the epitope. The paratope is a small region of the antibody's Fv region and contains parts of the antibody's heavy and light chains. Each arm of the Y shape of an antibody monomer is tipped with a paratope, which comprises the six CDRs, three light chain CDRs (LCDRs) and three heavy chains CDRs (HCDRs).

In the following, the antibody, or antigen-binding fragment thereof, of the present invention is defined by the structural features of its CDRs, in other words by the amino acid sequence of its HCDRs and/or LCDRs, or the amino acid structure of regions comprising the HCDRs and/or LCDRs. The skilled person will appreciate that minor variations, such as substitutions of one, two, three, four or even more amino acid residues, in the amino acid sequence may occur without affecting the functional properties, such as its binding capacity or binding affinity to BSSL, such as hBSSL, of the antibody, or antigen-binding fragment thereof. It will be understood that the first HCDR, second HCDR, third HCDR, first LCDR, second LCDR and third LCDR may be independently selected from the amino acid sequences recited.

In an embodiment, the antibody, or antigen-binding fragment thereof, comprises three complementarity determining regions (CDRs) of a heavy chain variable region (HCVR) (HCDRs) and three CDRs of a light chain variable region (LCVR) (LCDRs). In an embodiment, a first HCDR comprises, preferably consists of, an amino acid sequence according to SEQ ID NO: 7, a second HCDR comprises, preferably consists of, an amino acid sequence selected from the group consisting of SEQ ID NO: 8, 12 and 13 and a third HCDR comprises, preferably consists of, an amino acid sequence according to SEQ ID NO: 9. In an embodiment, a first LCDR comprises, preferably consists of, an amino acid sequence selected from the group consisting of SEQ ID NO: 10 and 14; and a third LCDR comprises, preferably consists of, an amino acid sequence according to SEQ ID NO: 11, 15 and 16.

It is believed that the second LCDR of the antibody, or the antigen-binding fragment thereof, is less involved in the binding to BSSL. However, in an embodiment, the antibody, or antigen-binding fragment thereof, comprises a second LCDR comprising, preferably consisting of, an amino acid sequence selected from the group consisting of ATS and AAS.

In an embodiment, the first HCDR comprises, preferably consists of, the amino acid sequence according to SEQ ID NO: 7, the second HCDR comprises, preferably consists of, the amino acid sequence according to SEQ ID NO: 8, the third HCDR comprises, preferably consists of, the amino acid sequence according to SEQ ID NO: 9, the first LCDR comprises, preferably consists of, the amino acid sequence according to SEQ ID NO: 10, the second LCDR comprises, preferably consists of, the amino acid sequence ATS, and the third LCDR comprises, preferably consists of, the amino acid sequence according to SEQ ID NO: 11.

In a particular embodiment, the HCVR comprises, preferably consists of, an amino acid sequence according to SEQ ID NO: 23. In another particular embodiment, the LCVR comprises, preferably consists of, an amino acid sequence selected from the group consisting of SEQ ID NO: 24 and 25. Hence, in an embodiment, the antibody, or the antigen-binding fragment thereof, comprises a HCVR comprising, preferably consisting of, an amino acid sequence according to SEQ ID NO: 23 and a LCVR comprising, preferably consisting of, an amino acid sequence according to SEQ ID NO: 24. In another embodiment, the antibody, or the antigen-binding fragment thereof, comprises a HCVR comprising, preferably consisting of, an amino acid sequence according to SEQ ID NO: 23 and a LCVR comprising, preferably consisting of, an amino acid sequence according to SEQ ID NO: 25.

In an embodiment, the antibody, or the antigen binding-fragment thereof, comprises a heavy chain (HC) comprising, preferably consists of, an amino acid sequence according to SEQ ID NO: 36 and/or a light chain (LC) comprising, preferably consisting of, an amino acid sequence according to SEQ ID NO: 37.

In another embodiment, the antibody, or the antigen binding-fragment thereof, comprises a HC comprising, preferably consisting of, an amino acid sequence according to SEQ ID NO: 38, and/or a LC comprising, preferably consisting of, an amino acid sequence according to SEQ ID NO: 39.

In an embodiment, the first HCDR comprises, preferably consists of, the amino acid sequence according to SEQ ID NO: 7, the second HCDR comprises, preferably consists of, the amino acid sequence according to SEQ ID NO: 12, the third HCDR comprises, preferably consists of, the amino acid sequence according to SEQ ID NO: 9, the first LCDR comprises, preferably consists of, the amino acid sequence according to SEQ ID NO: 10, the second LCDR comprises, preferably consists of, the amino acid sequence ATS, and the third LCDR comprises, preferably consists of, the amino acid sequence according to SEQ ID NO: 15.

In a particular embodiment, the HCVR comprises, preferably consists of, an amino acid sequence according to SEQ ID NO: 17. In another particular embodiment, the LCVR comprises, preferably consists of, an amino acid sequence according to SEQ ID NO: 18. Hence, in an embodiment, the antibody, or the antigen-binding fragment thereof, comprises a HCVR comprising, preferably consisting of, an amino acid sequence according to SEQ ID NO: 17 and a LCVR comprising, preferably consisting of, an amino acid sequence according to SEQ ID NO: 18.

In an embodiment, the antibody, or the antigen binding-fragment thereof, comprises a HC comprising, preferably consisting of, an amino acid sequence according to SEQ ID NO: 30 and/or a LC comprising, preferably consisting of, an amino acid sequence according to SEQ ID NO: 31.

In an embodiment, the first HCDR comprises, preferably consists of, the amino acid sequence according to SEQ ID NO: 7, the second HCDR comprises, preferably consists of, the amino acid sequence according to SEQ ID NO: 8, the third HCDR comprises, preferably consists of, the amino acid sequence according to SEQ ID NO: 9, the first LCDR comprises, preferably consists of, the amino acid sequence according to SEQ ID NO: 14, the second LCDR comprises, preferably consists of, the amino acid sequence AAS, and the third LCDR comprises, preferably consists of, the amino acid sequence according to SEQ ID NO: 11.

In a particular embodiment, the HCVR comprises, preferably consists of, an amino acid sequence according to SEQ ID NO: 19. In another particular embodiment, the LCVR comprises, preferably consists of, an amino acid sequence according to SEQ ID NO: 20. Hence, in an embodiment, the antibody, or the antigen-binding fragment thereof, comprises a HCVR comprising, preferably consisting of, an amino acid sequence according to SEQ ID NO: 19 and a LCVR comprising, preferably consisting of, an amino acid sequence according to SEQ ID NO: 20.

In an embodiment, the antibody, or the antigen binding-fragment thereof, comprises a HC comprising, preferably consisting of, an amino acid sequence according to SEQ ID NO: 32 and/or a LC comprising, preferably consisting of, an amino acid sequence according to SEQ ID NO: 33.

In an embodiment, the first HCDR comprises, preferably consists of, the amino acid sequence according to SEQ ID NO: 7, the second HCDR comprises, preferably consists of, the amino acid sequence according to SEQ ID NO: 13, the third HCDR comprises, preferably consists of, the amino acid sequence according to SEQ ID NO: 9, the first LCDR comprises, preferably consists of, the amino acid sequence according to SEQ ID NO: 14, the second LCDR comprises, preferably consists of, the amino acid sequence ATS, and the third LCDR comprises, preferably consists of, the amino acid sequence according to SEQ ID NO: 16.

In a particular embodiment, the HCVR comprises, preferably consists of, an amino acid sequence according to SEQ ID NO: 21. In another particular embodiment, the LCVR comprises, preferably consists of, an amino acid sequence according to SEQ ID NO: 22. Hence, in an embodiment, the antibody, or the antigen-binding fragment thereof, comprises a HCVR comprising, preferably consisting of, an amino acid sequence according to SEQ ID NO: 21 and a LCVR comprising, preferably consisting of, an amino acid sequence according to SEQ ID NO: 22.

In an embodiment, the antibody, or the antigen binding-fragment thereof, comprises a HC comprising, preferably consisting of, an amino acid sequence according to SEQ ID NO: 34 and/or a LC comprising, preferably consisting of, an amino acid sequence according to SEQ ID NO: 35.

In an embodiment, the first HCDR comprises, preferably consists of, the amino acid sequence according to SEQ ID NO: 49, the second HCDR comprises, preferably consists of, the amino acid sequence according to SEQ ID NO: 50 the third HCDR comprises, preferably consists of, the amino acid sequence according to SEQ ID NO: 51, the first LCDR comprises, preferably consists of, the amino acid sequence according to SEQ ID NO: 52 the second LCDR comprises, preferably consists of, the amino acid sequence according to SEQ ID NO: 53, and the third LCDR comprises, preferably consists of, the amino acid sequence according to SEQ ID NO: 15.

In a particular embodiment, the antibody, or the antigen binding-fragment thereof, comprises a HCVR consisting of SEQ ID NO: 26 and/or a LCVR consisting of SEQ ID NO: 27. A particular example of an antibody, or the antigen-binding fragment thereof, comprises a HCVR and LCVR pair comprising, preferably consisting of, the amino acid sequences according to SEQ ID NO: 26 and 27.

In a particular embodiment, the antibody, or the antigen binding-fragment thereof, comprises HC comprising, preferably consisting of, an amino acid sequence selected from the group consisting of SEQ ID NO: 28, 40, 42, and 47, and/or a LC comprising, preferably consisting of, an amino acid sequence selected from the group consisting of SEQ ID NO: 29, 41, 43, and 48. Particular examples of antibodies, or the antigen-binding fragments thereof, comprise a HC and LC pair comprising, preferably consisting of, the amino acid sequences according to SEQ ID NO: 28 and 29, 40 and 41, 42 and 43, 44 and 45, or 47 and 48.

Various modifications and/or additions can be made to an antibody, or antigen-binding fragment thereof, as disclosed herein in order to tailor the antibody, or antigen-binding fragment thereof, to a specific application without departing from the scope of the present disclosure. For example, the antibody, or antigen-binding fragment thereof, may have an amino acid sequence that has been extended by and/or comprises additional amino acids at the C-terminus and/or the N-terminus, for example at the C terminus and/or N terminus of its heavy or light chain. Thus, the antibody, or antigen-binding fragment thereof, may comprise any suitable number of additional amino acid residues, for example at least one additional amino acid residue. Each additional amino acid residue may individually or collectively be added in order to, for example, improve and/or simplify production, purification, stabilization in vivo or in vitro, coupling or detection of the antibody, or the antigen-binding fragment thereof. Such additional amino acid residues may comprise one or more amino acid residues added for the purpose of chemical coupling. An example is the addition of a cysteine residue. Additional amino acid residues may also provide a “tag” for purification or detection of the antibody, or antigen-binding fragment thereof, such as a Hiss tag, a (HisGlu)₃ tag, a “myc” (c-myc) tag or a FLAG tag.

The antibody, or antigen-binding fragment thereof, of the invention may be selected from full-length antibodies, combinations of CDR sequences, single-chain variable fragments, Fab fragments, F(ab′)₂ fragments, F(ab′)₃ fragments, Fab′ fragments, Fv fragments, dAb fragments, isolated complementarity determining regions (CDRs) and nanobodies although not limited thereto.

In an embodiment, the antibody, or antigen-binding fragment thereof is selected from the group consisting of a human antibody, a humanized antibody and a chimeric antibody, or an antigen-binding fragment thereof.

In an embodiment, the isolated antibody, or antigen-binding fragment thereof, has an isotype class selected from the group consisting of IgG, IgA, IgM, IgD and IgE. In a particular embodiment, the isotype class is IgG. For example, the isolated antibody, or antigen-binding fragment thereof, may be selected from the group consisting of isotype subclass IgG1 and IgG4.

It may be desirable to reduce or eliminate effector function by antibodies, or antigen-binding fragments thereof, for example, to prevent unwanted cytokine secretion. Other examples where reduced effector function may be warranted include preventing antibody-drug conjugates from interacting with Fc receptors (FcγRs) leading to off-target cytotoxicity.

Hence, in an embodiment, the antibody, or antigen-binding fragment thereof, comprises at least one Fc silencing mutation inhibiting interaction with FcγRs. For example, an antibody, or antigen-binding fragment thereof, based on IgG1 isotype class may comprise at least one, preferably at least two and more preferably all three of the Fc silencing mutations L234A, L235A and P329G.

Also, antibodies, or antigen-binding fragments thereof, of the IgG4 isotype are considered potential candidates for immunotherapy when reduced effector functions are desirable. IgG4 antibodies are known to be dynamic molecules able to undergo a process known as Fab arm exchange (FAE) and, without being bound by theory, this is thought to result in functionally monovalent, bispecific antibodies (bsAbs) with unknown specificity and, hence, potentially, reduced therapeutic efficacy. This may introduce undesired pharmacodynamics unpredictability for human immunotherapy.

Hence, in an embodiment, the isolated antibody, or antigen-binding fragment thereof comprises, at least one stabilizing mutation which prevents or reduces in vivo Fab arm exchange. For example, it has been suggested in the field that a single amino acid mutation (S228P) in the IgG4 core-hinge region is sufficient to prevent the in vivo FAE. In a particular embodiment, the isolated antibody, or antigen-binding fragment thereof, is of IgG4 isotype subclass and the at least one stabilizing mutation is S228P.

Currently preferred antibodies, or antigen-binding fragments thereof are denoted SL048-11 (heavy chain SEQ ID NO: 30 and light chain SEQ ID NO: 31), S-SL048-46 (heavy chain SEQ ID NO: 32 and light chain SEQ ID NO: 33), SL048-106 (heavy chain SEQ ID NO: 34 and light chain SEQ ID NO: 35), SOL-116 (heavy chain SEQ ID NO: 36 and light chain SEQ ID NO: 37), SL048-118 (heavy chain SEQ ID NO: 38 and light chain SEQ ID NO: 39), AS20 (heavy chain SEQ ID NO: 26 and light chain SEQ ID NO: 27), AS20 hIgG1 LALA-PG (heavy chain SEQ ID NO: 28 and light chain SEQ ID NO: 29), AS20 hIgG4 S228P (heavy chain SEQ ID NO: 40 and light chain SEQ ID NO: 41), CDR graft hIgG4 S228P (heavy chain SEQ ID NO: 42 and light chain SEQ ID NO: 43), AS20 IgG (heavy chain SEQ ID NO: 44 and light chain SEQ ID NO: 45) and chimeric AS20 IgG4 (Chi-AS20) (heavy chain SEQ ID NO: 47 and light chain SEQ ID NO: 48).

Suitable anti-BSSL antibodies, and antigen-binding fragments thereof, that can be used according to the invention are disclosed in WO 2021/010888, the teaching of which regarding anti-BSSL antibodies is hereby incorporated by reference.

In an embodiment, the antibody, or the antigen-binding fragment thereof, is an isolated antibody, or an isolated antigen-binding fragment thereof.

The present invention also relates to a pharmaceutical composition comprising an antibody, or an antigen-binding fragment thereof, according to the invention and a pharmaceutically acceptable carrier and/or excipient.

The term “pharmaceutical composition” refers to a preparation, which is in such form as to permit the biological activity of an active ingredient contained therein to be effective, and which contains no additional components which are unacceptably toxic to a subject to which the formulation would be administered.

The pharmaceutical composition may comprise a single type of antibody according to the invention or a single type of antigen-binding fragment according to the invention. Alternatively, the pharmaceutical composition may comprise a mixture of different types of antibodies according to the invention or different types of antigen-binding fragments according to the invention. It is also possible to have a pharmaceutical composition that comprises a mixture of at least one type of antibody according to the invention and at least one type of antigen-binding fragment according to the invention.

The antibody, or antigen-binding fragment thereof, or the pharmaceutical composition may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use, such as intravenous, subcutaneous or intramuscular infusion or injection, sterile aqueous or oily solutions or suspensions or sterile emulsions.

A “pharmaceutically acceptable excipient” as used herein refers to an excipient, which is nontoxic to a subject. Illustrative, but non-limiting, examples of such excipients include diluents, bulking agents, fillers, stabilizers, buffers, preservatives, adjuvants, antiadherents, binders, coatings, colors, disentegrants, flavors, glidants, lubricants, sorbents, and sweeteners.

A “pharmaceutically acceptable carrier” as used herein refers to a carrier, which is nontoxic to a subject. A pharmaceutically acceptable carrier includes, but is not limited to, vehicles, solvents, dispersion media, and the like. Preferably, the carrier is suitable for oral, intravenous, intramuscular, subcutaneous, or epidermal administration, e.g., by injection or infusion. Examples of suitable aqueous and non-aqueous carriers that may be employed in the pharmaceutical composition include water, ethanol, polyols (such as glycerol, propylene glycol, polyethylene glycol), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Other examples of aqueous carries that could be used according to the invention include saline and buffered solutions, such as phosphate-buffered saline (PBS).

Prevention of presence of microorganisms may be ensured both by sterilization procedures, and by the inclusion of various antibacterial and antifungal agents, for example, paraben, chlorobutanol, or phenol sorbic acid. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

In an example, the pharmaceutical composition may further comprise at least one additional active agent, such as at least two additional active agents, such as at least three additional active agents. Non-limiting examples of additional active agents that may prove useful in such combination are chemotherapeutics.

A further aspect of the invention relates to use of an antibody, or an antigen-binding fragment thereof, binding specifically to BSSL for the manufacture of a medicament for the treatment of a BSSL-expressing cancer in a subject. The antibody, for the antigen-binding fragment, thereof binds to an epitope present in an N-terminal part of BSSL ranging from a N-terminus of BSSL up to amino acid residue 300 in BSSL but excludes an active site of BSSL.

A BSSL-expressing cancer as used herein relates to a cancer with cancer cells expressing BSSL, or as the gene is named in humas, CEL. A BSSL-expressing cancer can thereby be identified by detecting presence of the BSSL protein in a culture medium, in which the cancer cells have been cultured, or in a biopsy from a tumor. Presence of BSSL in the culture medium or biopsy can be detected, for instance, using commercially available BSSL/BSDL ELISA kit, or using any of the antibodies of the invention, such as by labelling the antibodies of the invention with a detectable label, such as a fluorophore, a chemiluminescent label, an enzyme label, a gold label, or by detecting the antibodies or the invention with labelled secondary antibodies. Alternatively, a BSSL-expressing cancer can be identified by detecting CEL (BSSL) mRNA transcripts in cultured cancer cells or in a biopsy from a tumor.

A BSSL-expressing cancer is, as mentioned above, characterized by cancer cells expressing BSSL. In some such BSSL-expressing cancers, the corresponding non-cancerous cells, i.e., wild-type cells, of the particular tissue or organ do not express BSSL. For instance, in healthy subjects, BSSL is expressed in the exocrine pancreas and, in some species, such as humans, primates, dogs, cats and mice, BSSL is also expressed in lactating mammary gland. This means that BSSL is generally not expressed in, for instance, the colon, lung, prostate or liver in such healthy subject. However, BSSL expression has been detected in BSSL-expressing colon cancer, colorectal cancer, lung cancer, prostate cancer and liver cancer. Hence, in such BSSL-expressing cancers the wild-type cells in relevant healthy tissue or organ, such as colon, lung, prostate or liver, generally do not express BSSL, whereas cancer cells in BSSL-expressing colon cancer, colorectal cancer, lung cancer, prostate cancer and liver cancer express BSSL.

BSSL-expressing pancreatic cancer and breast cancer is characterized by higher BSSL expression in the pancreas and the breasts, respectively, as compared to the BSSL expression in the pancreas and the breasts, respectively, in healthy subjects not suffering from BSSL-expressing pancreatic cancer or BSSL-expressing breast cancer. Hence, such BSSL-expressing cancer is then characterized by higher BSSL expression and thereby higher BSSL quantity in the pancreas or the breasts as compared to healthy subjects.

For instance, a BSSL-expressing cancer is characterized by CEL (BSSL) mRNA transcripts per million of at least 25, preferably at least 30, such as at least 35, or at least 40 or even higher, such as at least 45 or at least 50. Correspondingly, cancer types having low or no BSSL expression have CEL (BSSL) mRNA transcripts per million of equal to or below 10, preferably equal to or below 5, such as equal to or below 4.5, or equal to or below 4 or even lower, such as equal to or below 3.5 or equal to or below 3. Examples of BSSL-expressing cancers and cancers that do not express BSSL are discussed in Example 2, see Table 3.

In an embodiment, a BSSL-expressing cancer has higher BSSL expression than the corresponding healthy tissue, from which the BSSL-expressing cancer is derived. For instance, a BSSL-expressing colon carcinoma has higher BSSL expression as compared to healthy, i.e., non-cancerous, colon epithelial cells. This should be compared to a cancer lacking BSSL or having low expression of BSSL, in which the BSSL expression is substantially the same as compared to the BSSL expression in the corresponding healthy tissue, from which the BSSL-expressing cancer is derived. Thus, a colon carcinoma that does not express BSSL or merely has low levels of BSSL expression typically has substantially the same BSSL expression as compared to healthy, i.e., non-cancerous, colon epithelial cells.

Illustrative, but non-limiting, examples of BSSL-expressing cancers include BSSL-expressing colon cancer, lung cancer, colorectal cancer, pancreatic cancer, breast cancer, prostate cancer and liver cancer.

The present document is also directed to a method for treating a BSSL-expressing cancer in a subject. This method comprises administering a therapeutically effective amount of an antibody, or antigen-binding fragment thereof, or a pharmaceutical composition to the subject in need thereof. The antibody, or the antigen-binding fragment thereof, binds to an epitope present in an N-terminal part of BSSL ranging from a N-terminus up to amino acid residue 300 in BSSL but excludes an active site of BSSL.

As used herein, the terms “treatment” or “treat” include both prophylactic or preventive treatment (that prevent and/or slow the development of a cancer disease) and curative, therapeutic or disease-modifying treatment, including therapeutic measures that cure, slow down, lessen symptoms of, and/or halt progression of a cancer disease; and treatment of subjects at risk of contracting a cancer disease or suspected to have contracted a cancer disease, as well as subjects who are ill or have been diagnosed as suffering from a cancer disease. The term does not necessarily imply that a subject is treated until total recovery. The terms “treatment” and “treat” further include reducing the risk of developing a cancer disease, e.g., in the form of preventive treatment. The terms “treatment” and “treat” are also intended to include the potentiation or otherwise enhancement of one or more primary prophylactic or therapeutic measure. Treatment therefore encompass clinical intervention in an attempt to alter the natural course of the cancer diseases of the subject being treated, and can be performed either for prophylaxis or during the course of clinical pathology. Desirable effects of treatment include, but are not limited to, preventing occurrence or recurrence of the cancer disease, alleviation of symptoms (improvement of quality of life), diminishment of any direct or indirect pathological consequences of the cancer disease, preventing metastasis, reducing primary tumor size, decreasing the rate of disease progression, amelioration or palliation of the disease state, and remission or improved prognosis. An antibody, or antigen-binding fragment thereof, according to the present invention may be used to delay development of a cancer disease or to slow the progression of a cancer disease. By “reduce” or “inhibit” is meant the ability to cause an overall decrease of 20%, 30%, 40%, 50%, 60%, 70%, 75%, 80%, 85%, 90%, 95%, or greater. Reduce or inhibit can refer to the symptoms of the cancer disease being treated. Reduce or inhibit also encompass delaying the onset of a cancer disease.

The treatment using an antibody, or an antigen-binding fragment thereof, or a pharmaceutical composition as disclosed herein is typically a passive immunotherapy, meaning that an antibody, or antigen-binding fragment thereof, or a pharmaceutical composition comprising such an antibody, or antigen-binding fragment thereof, is administered to a subject in need thereof.

A subject according to the present disclosure may be any human or non-human animal. The term “non-human animal” includes all vertebrates, e.g., mammals and non-mammals, such as non-human primates, sheep, dogs, cats, horses, cows, chickens, amphibians, reptiles, etc. Mammals include, but are not limited to, domesticated animals, e.g., cows, sheep, cats, dogs, and horses, primates, e.g., humans and non-human primates, such as monkeys, rabbits, and rodents, e.g., mice and rats. The term “subject” may be used interchangeably with the term “patient” in the present document. The subject may be a human.

An antibody, or antigen-binding fragment thereof, or a pharmaceutical composition according to the invention may be administered in standard manner for the condition that it is desired to treat, for example by oral, topical, parenteral, intravenous, subcutaneous, buccal, nasal, or rectal administration or by inhalation. For example, the antibody, or antigen-binding fragment thereof, or the pharmaceutical composition may be formulated for parenteral administration, such as intravenous or subcutaneous administration, in particular subcutaneous administration.

Typically, the antibody, or antigen-binding fragment thereof, or the pharmaceutical composition is systemically administered. The administration mode may e.g., be parenteral, such as by intravenous or subcutaneous administration, in particular subcutaneous administration. However, also local administration, such as intratumoral administration, can be used instead of, or as a complement to, systemic administration.

Although the administration regimen may be adjusted to the particular cancer disease and subject to be treated, typically the antibody, or antigen-binding fragment thereof, or the pharmaceutical composition is administered 1-3 times per week, such as 1-2 times per week, such as 1 time a week although other administration regimes are also possible.

The antibodies, or antigen-binding fragments thereof, or the pharmaceutical compositions may also be administered in combination therapy, i.e., combined with other agents. For example, the combination therapy can include an antibody, or antigen-binding fragment thereof, according to the invention, combined with at least one chemotherapeutic. It will be understood that the combination therapy encompasses sequential as well as concurrent administration. The term “concurrent” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

Alternatively, the combination therapy may include an antibody, or antigen-binding fragment thereof, according to the invention combined with another anti-cancer treatment, such as radio therapy or surgery.

Dosage regimens may be adjusted to provide the optimum desired response, e.g., a therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage.

The therapeutically effective amount of an antibody, or antigen-binding fragment thereof, may vary according to factors such as the disease state, age, sex, and weight of the subject, and the ability of the antibody, or antigen-binding fragment thereof, to elicit a desired response in the subject. A therapeutically effective amount is also one, in which any toxic or detrimental effects of the administered antibody, of antigen-binding fragment thereof, are outweighed by the therapeutically beneficial effects. When the antibody, or antigen-binding fragment thereof, is used for the prevention of a condition (prophylactic purposes), typically but not necessarily, the prophylactically effective amount is less than the therapeutically effective amount as the prophylactic dose is used in subjects prior to or at an earlier stage of the cancer disease.

A pharmaceutically effective amount, i.e., the dose, of an antibody, or antigen-binding fragment thereof, according to the invention is typically in the range of from about 0.0001 to 100 mg/kg, and more usually 0.01 to 5 mg/kg, of the subject body weight. However, the exact dose is preferably adjusted depending on e.g., the cancer disease to be treated or prevented, the age and/or the sex of the subject and whether it is intended to treat or prevent a condition.

### EXAMPLES

### Example 1—Evaluation of the Effects of SOL-116 on Primary Tumor and Metastasis from Four Different Indications in ZTX®-ONCOLEADS

The purpose of this Example was to determine the possible effects of SOL-116 on primary tumor size and on formation of metastasis. Four different cell lines were used and anti-cancer efficacy of SOL-116 was determined as the change in primary tumor size, i.e., tumor growth or reduction, and the number of tumor cells disseminated to the distal caudal venous plexus (CVP) three days after implantation.

### Study Design

The example investigated four different cell lines covering four different cancer indications. A human IgG4 antibody (human IgG4 S241P standard (Abzena)) was used for control. SOL-116 was evaluated for efficacy on primary tumor size and was evaluated directly after implantation and at 3 days after implantation. Evaluation of metastasis was made at 3 days after implantation.

TABLE 1
Description of groups for analysis in
ZTX ®-ONCOLEADS platform

| | | | | Group |
|---|---|---|---|---|
| Tumor cell line | Indication | Treatment | Concentration | size |
| HTC 116 | Colorectal | Isotype IgG4 | 10 mg/L | 20 |
| | | SOL-116 | | |
| PC3 | Prostate | Isotype IgG4 | | |
| | | SOL-116 | | |
| BT-549 | Breast | Isotype IgG4 | | |
| | | SOL-116 | | |
| HPAF-II | Pancreatic | Isotype IgG4 | | |
| | | SOL-116 | | |

### Zebrafish Tumor Xenograft (ZTX) Model

Transgenic Tg(Fli1:EGFP)y1 zebrafish embryos were raised at 28° C. for 48 hours in E3 embryo medium with 0.2 mM 1-phenyl-2-thiourea (PTU). Unfertilized eggs or larvae that did not appear healthy or exhibited any obvious developmental defects were excluded prior to treatment onset.

Approximately 500 Dil-labeled cancer cells were co-injected subcutaneously into the perivitelline space of 2 days old larvae with human IgG4 S241P control antibody at 10 mg/L or SOL-116 at 10 mg/L. Larvae, in which tumor cells had been invertedly injected into circulation, or larvae with erroneous implantation of the tumor in the yolk rather than the perivitelline space were excluded from the study. Selected tumor-bearing embryos were sorted into experimental groups (20 embryos/group) and pictures of primary tumors were taken right after injection. Tumor-bearing embryos were incubated in E3+PTU medium during 72 hours at 36° C. After incubation, pictures of the primary tumors and the CVP were taken. Larvae that died or were lost by other means during the study were excluded from the final analysis.

### Analysis of Tumor Growth Inhibition and Metastasis

Images obtained right after implantation (day 0) and after 72 hours incubation (day 3) were analyzed using an in-house developed software (BioReperia). Tumor growth regression was calculated by dividing the number of tumor pixels at day 3 by the number of tumor pixels at day 0 in the same embryo and multiplied by 100.

### Statistical Analysis

Data is presented as mean±standard error of the mean (SEM) and Student's t-test was used to determine the p-value, differences corresponding to a value <0.05 are denoted significant. Experimental data was analyzed using GraphPad.

### Results

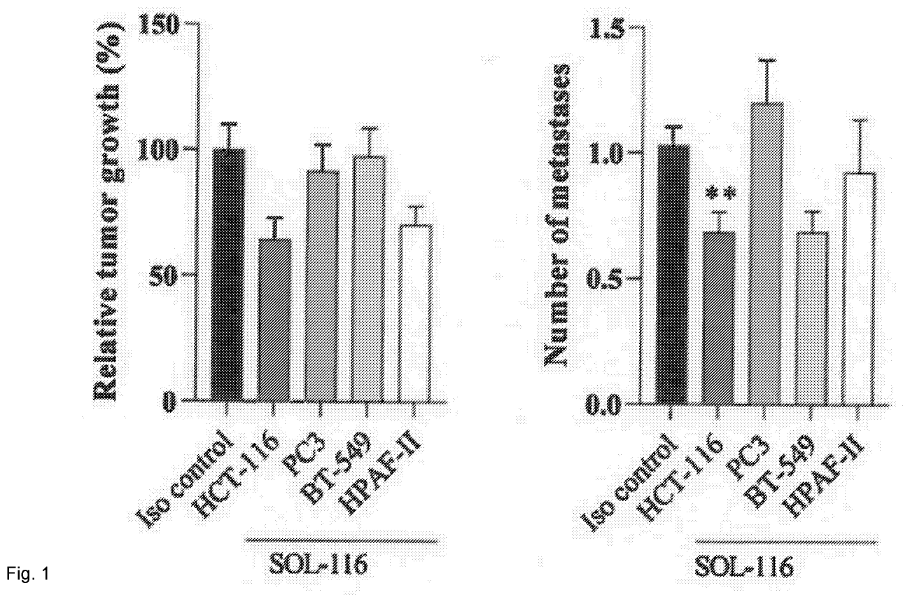

All analyzed cell lines successfully engrafted and formed metastasis in the zebrafish embryos. FIG. 1 illustrates compiled results for all cell lines analyzed for both effect on primary tumor and metastasis normalized to the human IgG4 S241P isotype control (Iso control). SOL-116 treatment decreased HCT 116 primary tumor growth compared to human IgG4 S241P isotype control (Iso control) after 3 days of treatment although not significant. SOL-116 treatment also significantly decreased the number of distant metastases observed in the CVP site. SOL-116 did not have any significant effect on PC3 primary tumor growth or number of distant metastases compared to human IgG4 S241P isotype control (Iso control) after 3 days of treatment. SOL-116 did not have any significant effect on BT-549 primary tumor growth but a slight, non-significant, decrease in the number of distant metastases compared to human IgG4 S241P isotype control (Iso control) after 3 days of treatment. SOL-116 treatment decreased HPAF-II primary tumor growth compared to human IgG4 S241P isotype control (Iso control) after 3 days of treatment although not significant. However, SOL-116 treatment had no effect on the number of distant metastases.

The HCT-116 colorectal cancer cell line is the highest BSSL cancer cell line of the four cancer cell lines investigated in the present Example.

### Example 2—Evaluation of the Effects of SOL-116 on Primary Tumor and Metastasis of PDX Models in ZTX®-ONCOLEADS

The purpose of this Example was to determine the anti-cancer efficacy of SOL-116 on primary tumor size and on formation of metastasis of lung and colorectal PDX cancer models. The anti-cancer efficacy of SOL-116 was determined as the change in primary tumor size, i.e., tumor growth or reduction, and the number of tumor cells disseminated to the distal CVP three days after implantation.

### Study Design

The Example was composed of six different PDX models covering two different cancer indications. Three of the models were expressing high levels of BSSL mRNA and three were expressing none or low levels (Table 3). Vehicle (phosphate buffered saline, PBS) was used as negative control. The same isotype control and anti-BSSL antibody (SOL-116) as in Example 1 was used in this Example. Evaluation of primary tumor sizes were made directly after implantation and at 3 days after. Evaluation of metastasis were made at 3 days after implantation.

TABLE 2
Description of groups for analysis in
ZTX ®-ONCOLEADS platform

| | | BSSL | | | |
|---|---|---|---|---|---|
| PDX | Indica- | expres- | | Concen- | Group |
| model | tion | sion | Treatment | tration | size |
| CXF-1788 | Colon | High | Negative control | — | 20 |
| | | | Isotype IgG4 | 10 mg/L | |
| | | | SOL-116 | | |
| CXF-158 | | | Negative control | | |
| | | | Isotype IgG4 | 10 mg/L | |
| | | | SOL-116 | | |
| CXF-2836 | | Low | Negative control | — | |
| | | | Isotype IgG4 | 10 mg/L | |
| | | | SOL-116 | | |
| CXF-533 | | | Negative control | — | |
| | | | Isotype IgG4 | 10 mg/L | |
| | | | SOL-116 | | |
| LXFS-1129 | Lung | High | Negative control | — | |
| | | | Isotype IgG4 | 10 mg/L | |
| | | | SOL-116 | | |
| LXFS-2156 | | Low | Negative control | — | |
| | | | Isotype IgG4 | 10 mg/L | |
| | | | SOL-116 | | |

TABLE 3
BSSL expression

| PDX model | CEL (BSSL) mRNA transcripts per million |
|---|---|
| CXF-1788 | 404.95 |
| CXF-158 | 57.06 |
| CXF-2836 | 96.58 |
| CXF-533 | 3.03 |
| LXFS-1129 | 2.00 |
| LXFS-2156 | 0.07 |

### Zebrafish Tumor Xenograft (ZTX) Model

Transgenic Tg (Fli1: EGFP) y1 zebrafish embryos were raised at 28° C. for 48 hours in E3 embryo medium with 0.2 mM PTU. Unfertilized eggs or larvae that did not appear healthy or exhibited any obvious developmental defects were excluded prior to treatment onset.

Approximately 700 Dil-labeled cancer cells were co-injected subcutaneously into the perivitelline space of 2 days old larvae with human IgG4 S241P control antibody at 10 mg/L or SOL-116 at 10 mg/L. Larvae, in which tumor cells had been invertedly injected into circulation, or larvae with erroneous implantation of the tumor in the yolk rather than the perivitelline space were excluded from the study. Selected tumor-bearing embryos were sorted into experimental groups (20 embryos/group) and pictures of primary tumors were taken right after injection. Tumor-bearing embryos were incubated in E3+PTU medium during 72 hours at 36° C. After incubation, pictures of the primary tumors and the CVP were taken. Larvae that died or were lost by other means during the study were excluded from the final analysis.

### Analysis of Tumor Growth Inhibition and Metastasis

Images obtained right after implantation (day 0) and after 72 hours incubation (day 3) were analyzed using an in-house developed software (BioReperia). Tumor growth regression was calculated by dividing the number of tumor pixels at day 3 by the number of tumor pixels at day 0 in the same embryo and multiplied by 100.

### Statistical Analysis

Data is presented as mean±standard error of the mean (SEM) and Student's t-test was used to determine the p-value, differences corresponding to a value <0.05 are denoted significant. Experimental data was analyzed using GraphPad.

### Results

### LXS-1129

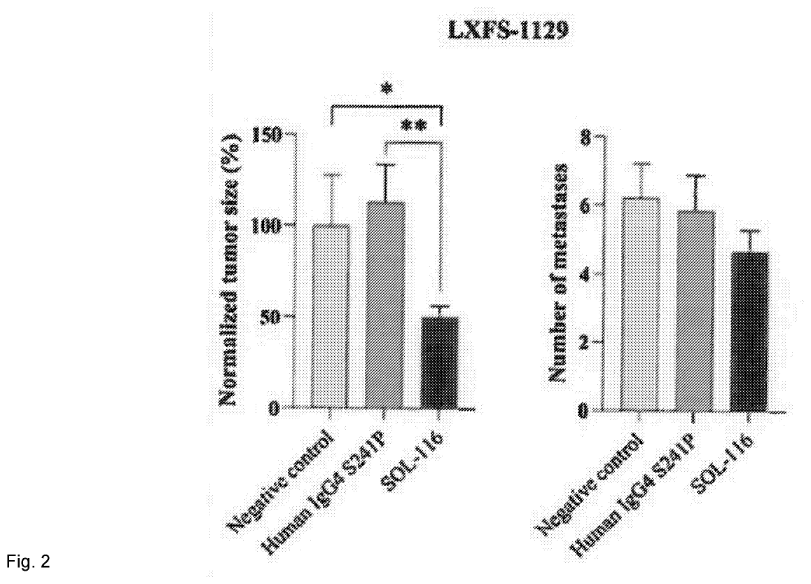

FIG. 2 shows that SOL-116 significantly reduced primary tumor growth compared to the negative control group and the human IgG4 S241P isotype control group after 3 days of treatment. SOL-116 treatment resulted in a non-significant reduction in the number of distant metastases observed in the CVP site.

### CXF-1788

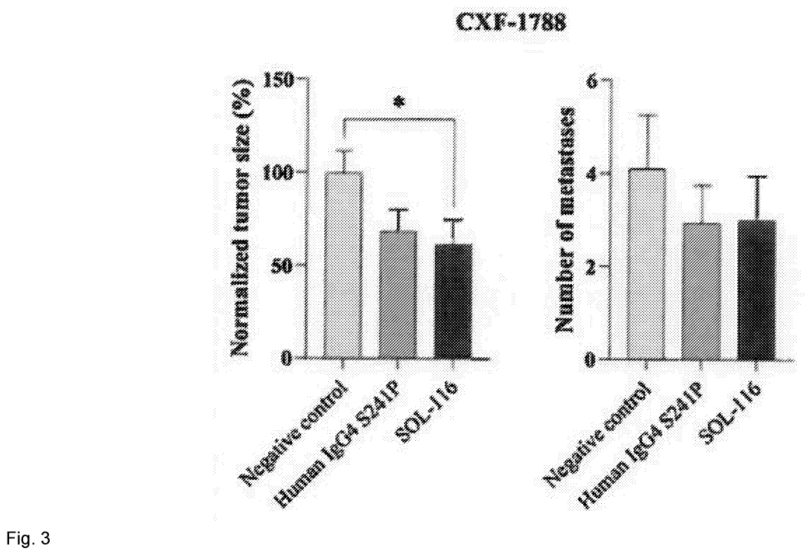

FIG. 3 shows that SOL-116 significantly reduced the primary tumor size compared to the negative control group and the human IgG4 S241P isotype control group after 3 days of treatment. The number of distant metastases compared to the human IgG4 S241P isotype control group was not affected by SOL-116 after 3 days of treatment.

### CXF-158

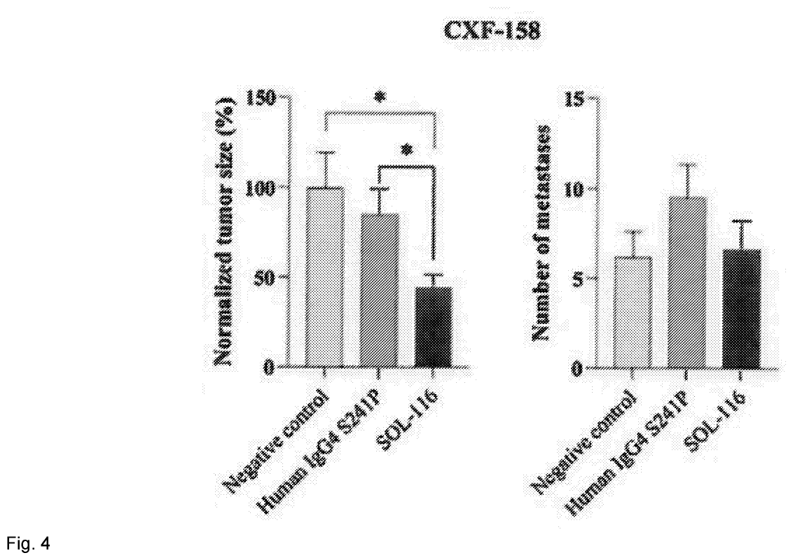

FIG. 4 shows that SOL-116 significantly affected CXF-158 primary tumor growth compared to the negative control group and human IgG4 S241P isotype control group. SOL-116 did not affect the number of distant metastases compared to the negative control group after 3 days of treatment.

### LXFS-2156

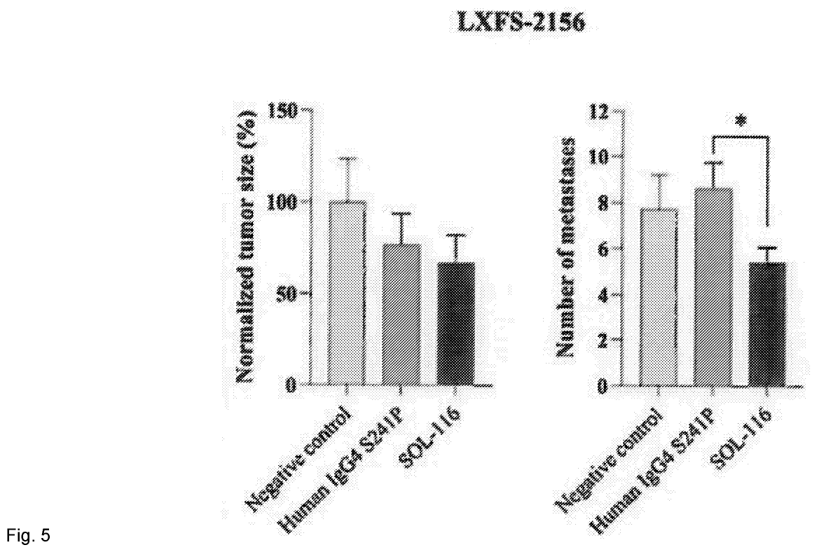

FIG. 5 shows that SOL-116 had no significant effect on LXFS-2156 primary tumor growth. However, it significantly affected the number of distant metastases compared to the human IgG S241P isotype control after 3 days of treatment.

### CXF-2836

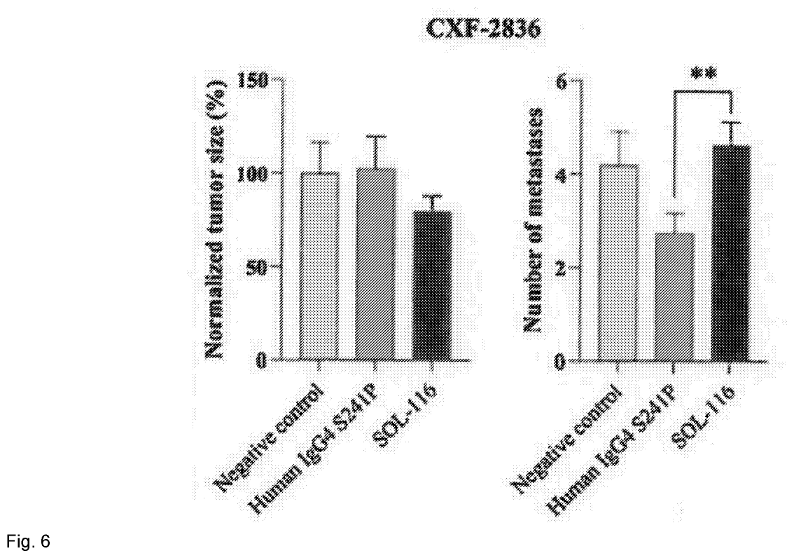

FIG. 6 shows that SOL-116 had no significant effect on CXF-2836 primary tumor growth compared to the negative control group of the human IgG S241P isotype control group. SOL-116 significantly increased the number of distant metastases compared to the human IgG4 S241P isotype control group after 3 days of treatment.

However, significant changes in the number of metastasized cancer cells observed in the CXF-2836 PDX model were considered to not be biologically relevant due to the low metastatic capability exhibited by the model. In order to evaluate the anti-metastatic efficacy of drugs in the ZTX® model higher than five disseminated cancer cells in the CVP in average should be observed.

### CXF-533

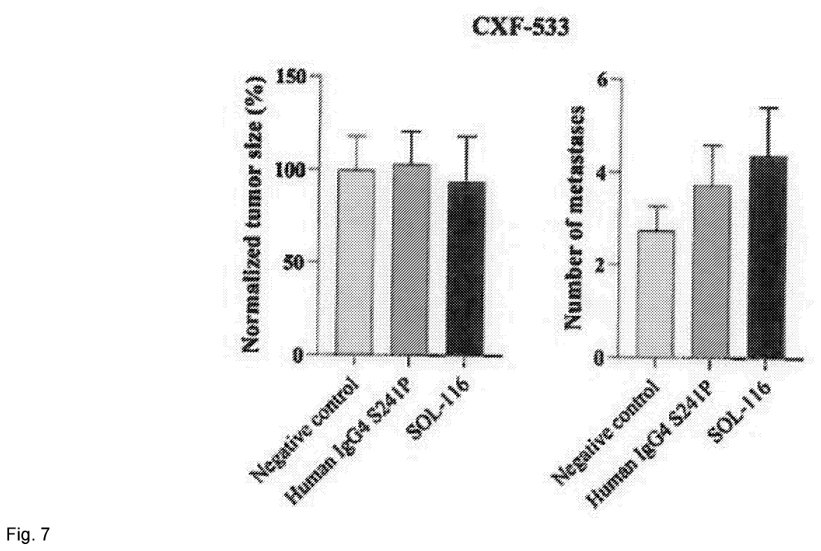

FIG. 7 shows that SOL-116 had no significant effect on CXF-533 primary tumor growth or the number of distant metastases compared to the negative control group and/or the human IgG4 S241P isotype control after 3 days of treatment.

### Conclusion

SOL-116 had anti-tumor efficacy on LXFS-1129, CXF-1788 and CXF-158 PDX models, which correlated to their high BSSL expression levels. SOL-116 did not show any anti-tumor effect on LXFS-2156, CXF-533 and CXF-2836 PDX models, which correlated to their ow BSSL expression levels.

### Example 3—Evaluation of the Effects of SOL-116 and Other Anti-BSSL Antibodies on Primary Tumor and Metastasis of PDX Models in ZTX®-ONCOLEADS

The purpose of this Example was to determine the anti-cancer efficacy of SOL-116 and other anti-BSSL antibodies on primary tumor size and on formation of metastasis of lung and colorectal PDX cancer models with high or low expression of BSSL. The anti-cancer efficacy was determined as the change in primary tumor size, i.e., tumor growth or reduction, and the number of tumor cells disseminated to the distal CVP three days after implantation.

Study Design The Example was composed of four different PDX models covering two different cancer indications. Two of the models were expressing high levels of BSSL mRNA and two were expressing none or low levels (Table 4). An irrelevant human IgG4 antibody was used for isotype control. Vehicle (phosphate buffered saline, PBS) was used as negative control. Evaluation of primary tumor sizes was made directly after implantation and at 3 days after. Evaluation of metastasis was made at 3 days after implantation.

TABLE 4
Description of groups for analysis in
ZTX ®-ONCOLEADS platform

| | | | | | |
|---|---|---|---|---|---|
| | | BSSL | | | |
| PDX | Indica- | expres- | | Concen- | Group |
| model | tion | sion | Treatment | tration | size |

| | | | | |
|---|---|---|---|---|
| CXF-158 | Colon | High | Negative control | |
| | | | Isotype IgG4 | 10 mg/L |
| | | | SOL-116 | |
| | | | Chi-AS20 | |
| | | | SL048-46 | |
| CXF-533 | | Low | Negative control | |
| | | | Isotype IgG4 | 10 mg/L |
| | | | SOL-116 | |
| | | | Chi-AS20 | |
| | | | SL048-46 | |
| LXFS-1129 | Lung | High | Negative control | |
| | | | Isotype IgG4 | 10 mg/L |
| | | | SOL-116 | |
| | | | Chi-AS20 | |
| | | | SL048-46 | |
| LXFS-2156 | | Low | Negative control | |
| | | | Isotype IgG4 | 10 mg/L |
| | | | SOL-116 | |
| | | | Chi-AS20 | |
| | | | SL048-46 | |

### Zebrafish Tumor Xenograft (ZTX) Model

Transgenic Tg (Fli1: EGFP) y1 zebrafish embryos were raised at 28° C. for 48 hours in E3 embryo medium with 0.2 mM PTU. Unfertilized eggs or larvae that did not appear healthy or exhibited any obvious developmental defects were excluded prior to treatment onset.

PDX tumor tissue was dissociated and single-cell suspension was labelled with Dil red fluorescent dye. Approximately 700 Dil-labeled cancer cells were co-injected subcutaneously into the perivitelline space of 2 days old larvae with±human IgG4 B1-8 isotype, ±SOL-116, ±Chia-AS20 or ±SL048-46 antibodies at 10 mg/L. Larvae, in which tumor cells had been invertedly injected into circulation, or larvae with erroneous implantation of the tumor in the yolk rather than the perivitelline space were excluded from the study. Selected tumor-bearing embryos were sorted into experimental groups (20 embryos/group) and pictures of primary tumors were taken right after injection. Tumor-bearing embryos were incubated in E3+PTU medium during 72 hours at 36° C. After incubation, pictures of the primary tumors and the CVP were taken. Larvae that died or were lost by other means during the study were excluded from the final analysis.

### Analysis of Tumor Growth Inhibition and Metastasis

Images obtained right after implantation (day 0) and after 72 hours incubation (day 3) were analyzed using an in-house developed software (BioReperia). Tumor growth regression was calculated by dividing the number of tumor pixels at day 3 by the number of tumor pixels at day 0 in the same embryo and multiplied by 100. The number of disseminated cancer cells to the CVP site was counted manually.

### Statistical Analysis

Data is presented as mean±SEM and Student's t-test was used to determine the p-value, differences corresponding to a value <0.05 are denoted significant. Experimental data was analyzed using GraphPad.

### Results

### LXFS-1129

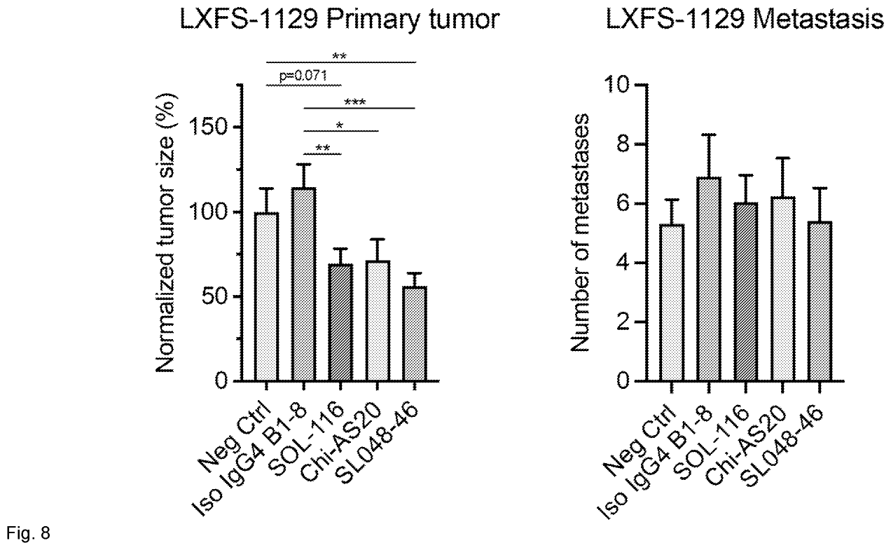

FIG. 8 shows that treatment with SL048-46 antibodies significantly reduced primary tumor size compared to the negative control group and the isotype IgG4 B1-8 control group after 3 days of treatment. Meanwhile, treatment with SOL-116 and Chi-AS20 antibodies significantly reduced primary tumor size compared to the isotype IgG4 B1-8 control group after 3 days of treatment.

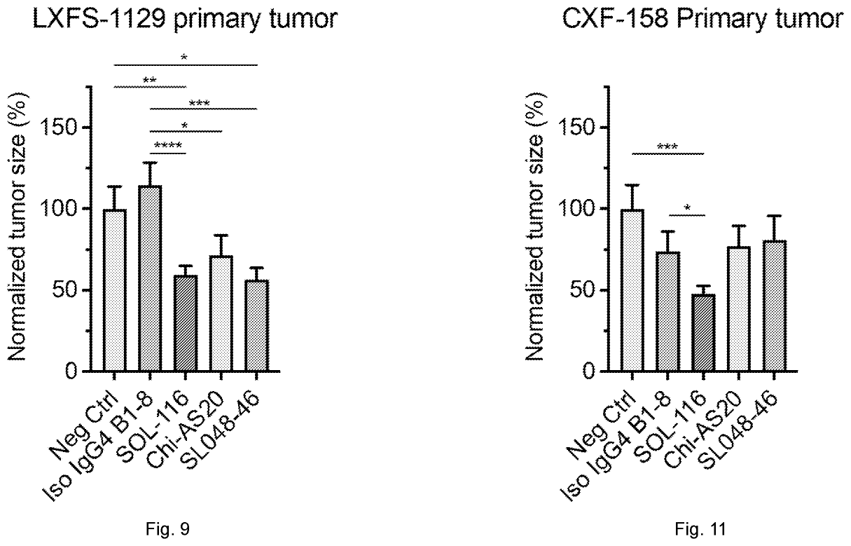

FIG. 8 shows that SOL-116 antibody treatment showed a borderline significance in the reduction of primary tumor size compared to the negative control group (p=0.071). Furthermore, this effect reached a mathematical significance when the data were compiled with Example 2 (FIG. 9).

### CXF-158

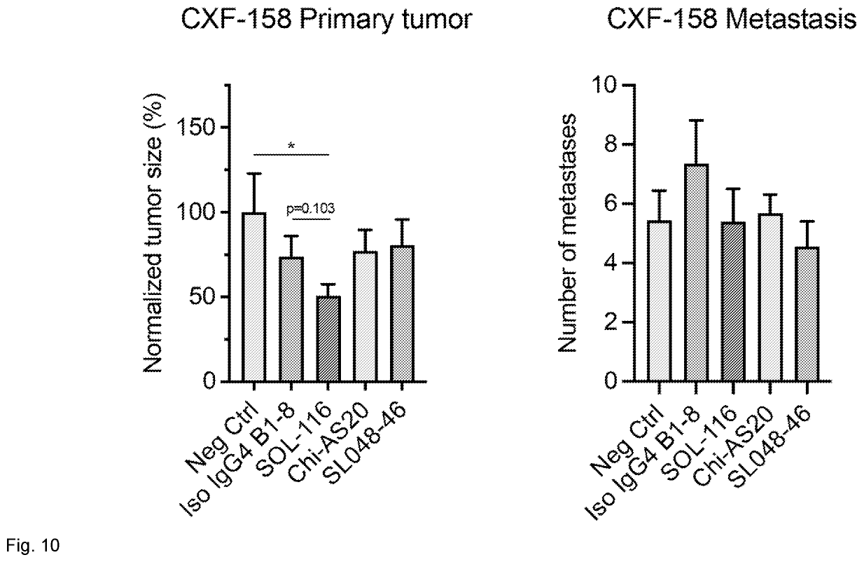

FIG. 10 shows that treatment with SOL-116 significantly reduced the primary tumor size compared to the negative control group after 3 days of treatment. SOL-116 treatment showed no effect on primary tumor size compared to the isotype IgG4 B1-8 control group (FIG. 10). However, SOL-116 significantly reduced the primary tumor size compared to the isotype IgG4 B1-8 control group when tumor data were compiled (FIG. 11)

### LXFS-2156

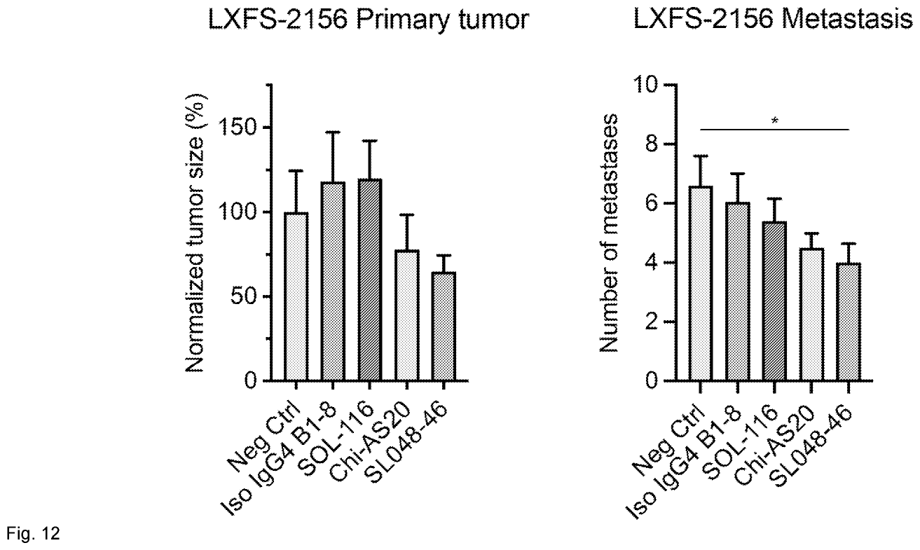

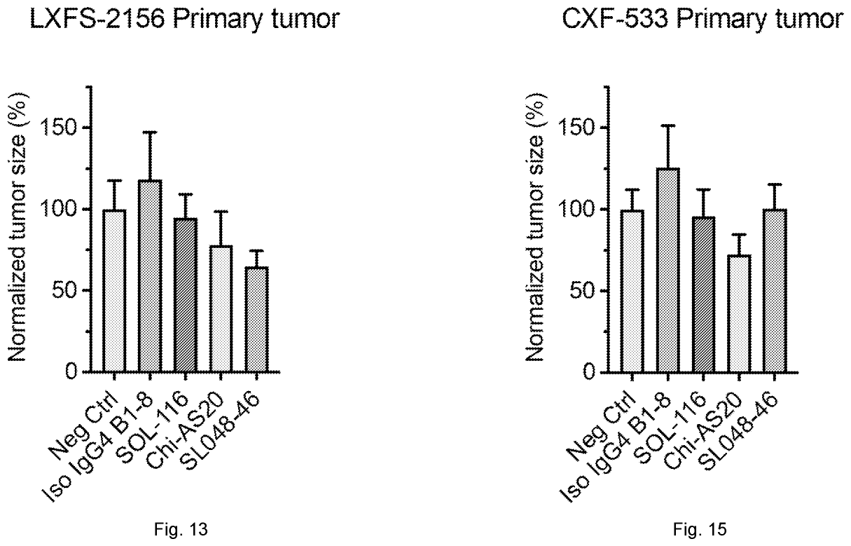

FIG. 12 shows that treatment with SOL-116, Chi-AS20, or SL048-46 antibodies had no significant effect on primary tumor size compared to the negative control or the isotype IgG4 B1-8 control group after 3 days of treatment, and this effect was not altered when negative control and SOL-116 data were compiled (FIG. 13).

FIG. 12 also shows that treatment with SL048-46 antibody significantly reduced the number of disseminated tumor cells to the CVP compared to the negative control group after 3 days of treatment; meanwhile, treatment with SOL-116 or Chi-AS20 antibodies showed no effect on tumor cell dissemination (FIG. 12).

### CXF-533

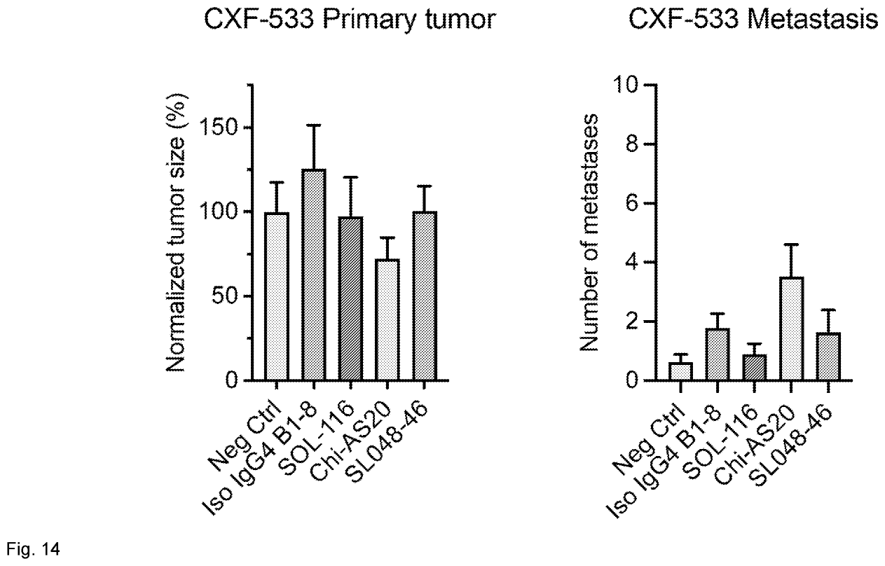

FIG. 14 shows that treatment with SOL-116, Chi-AS20, or SL048-46, antibodies had no significant effect on primary tumor size compared to the negative control or the isotype IgG4 B1-8 control group after 3 days of treatment, and this effect was not altered when negative control and SOL-116 data were compiled (FIG. 14). FIG. 15 also shows that treatment with SOL-116, Chi-AS20, SL048-46, or CDR graft antibodies did not affect the number of disseminated tumor cells to the CVP site after 3 days of treatment.

### Conclusions

We conclude that the anti-tumor efficacy and specificity of SOL-116 from Example 2 have been verified. SOL-116 antibodies have anti-tumor efficacy on lung and colorectal cancer PDX models, LXFS-1129 and CXF-158, which correlate to their high BSSL expression status. Meanwhile, Chi-AS20 and SL048-46 antibodies have anti-tumor efficacy only on the lung cancer LXFS-1129 model and not on the colorectal cancer CXF-158 model, which shows their restricted anti-tumor effect. None of the tested antibodies showed any anti-tumor effect on LXFS-2156 and CXF-533 PDX models, which correlate to their low expression of BSSL.

TABLE 5
Listing of the sequences and SEQ ID NO:s
used in the present document

| SEQ | | |
|---|---|---|
| ID | | |
| NO: | Nomenclature | Sequence |
| 1 | BSSL Epitope | YTEGGF |
| | 7-12 | |
| 2 | BSSL Epitope | LENPQPHPGWQGTL |
| | 42-55 | |
| 3 | BSSL Epitope | AKLGAVYTEGGF |
| | 1-12 | |
| 4 | BSSL Epitope | NIWVPQGRKQVSRDLPVM |
| | 84-101 (SL048- | |
| | 46) | |
| 5 | BSSL Epitope | VKRNIAA |
| | 174-180 (SOL- | |
| | 116) | |
| 6 | BSSL Epitope | HYVGFVPVIDGDF |
| | 283-295 | |
| | (SL048-11) | |
| 7 | HCDR1 | GYTFTSYN |
| | (SL048-11, | |
| | SL048-46, | |
| | SL048-106, | |
| | SOL-116, | |
| | SL048-118) | |
| 8 | HCDR2 | INPGDGAT |
| | (SL048-46, | |
| | SOL-116, | |
| | SL048-118) | |
| 9 | HCDR3 | ARDYYGSSPLGY |
| | (SL048-11, | |
| | SL048-46, | |
| | SL048-106, | |
| | SOL-116, | |
| | SL048-118) | |
| 10 | LCDR1 (SL048- | PSISY |
| | 11, SOL-116, | |
| | SL048-118) | |
| 11 | LCDR3 (SL048- | HQRSSSPT |
| | 46, SOL-116, | |
| | SL048-118) | |
| 12 | HCDR2 | IYPGDGAT |
| | (SL048-11) | |
| 13 | HCDR2 | IYPGDGST |
| | (SL048-106) | |
| 14 | LCDR1 (SL048- | SSISY |
| | 46, SL048-106) | |
| 15 | LCDR3 (SL048- | HQRSSYPT |
| | 11 + AS20) | |
| 16 | LCDR3 (SL048- | HQRSSTPT |
| | 106) | |
| 17 | SL048-11 | QVQLVQSGAEVKKPGASVKVSCKASGYTFTSYNMHWVRQAPGQGL |
| | HCVR | EWMGVIYPGDGATSYAQKFKGRVTMTRDTSTSTVYMELSSLRSED |
| | | TAVYYCARDYYGSSPLGYWGQGTLVTVSS |
| 18 | SL048-11 | DIQMTQSPSSLSASVGDRVTITCSASPSISYMNWYQQKPGKAPKL |
| | LCVR | LIYATSSLASGVPSRFSGSGSGTDFTLTISSLQPEDFATYYCHQR |
| | | SSYPTFGQGTKLEIK |
| 19 | SL048-46 | QVQLVQSGAEVKKPGASVKVSCKASGYTFTSYNMHWVRQAPGQGL |
| | HCVR | EWMGVINPGDGATSYNQKFQGRVTMTRDTSTSTVYMELSSLRSED |
| | | TAVYYCARDYYGSSPLGYWGQGTLVTVSS |
| 20 | SL048-46 | DIQMTQSPSSLSASVGDRVTITCRASSSISYLNWYQQKPGKAPKL |
| | LCVR | LIYAASSLASGVPSRFSGSGSGTDFTLTISSLQPEDFATYYCHQR |
| | | SSSPTFGQGTKLEIK |
| 21 | SL048-106 | QVQLVQSGAEVKKPGASVKVSCKASGYTFTSYNMHWVRQAPGQGL |
| | HCVR | EWIGVIYPGDGSTSYNQKFQGRVTMTRDTSTSTVYMELSSLRSED |
| | | TAVYYCARDYYGSSPLGYWGQGTLVTVSS |
| 22 | SL048-106 | DIQMTQSPSSLSASVGDRVTITCSASSSISYMNWYQQKPGKAPKL |
| | LCVR | LIYATSKLPSGVPSRFSGSGSGTDFTLTISSLQPEDFATYYCHQR |
| | | SSTPTFGQGTKLEIK |
| 23 | SOL-116 and | QVQLVQSGAEVKKPGASVKVSCKASGYTFTSYNMHWVRQAPGQGL |
| | SL048-118 | EWMGVINPGDGATSYAQKFKGRVTMTRDTSTSTVYMELSSLRSED |
| | HCVR | TAVYYCARDYYGSSPLGYWGQGTLVTVSS |
| 24 | SOL-116 LCVR | DIQMTQSPSSLSASVGDRVTITCRASPSISYMNWYQQKPGKAPKL |
| | | LIYATSSLASGVPSRFSGSGSGTDFTLTISSLQPEDFATYYCHQR |
| | | SSSPTFGQGTKLEIK |
| 25 | SL048-118 | DIQMTQSPSSLSASVGDRVTITCSASPSISYMNWYQQKPGKAPKL |
| | LCVR | LIYATSSLPSGVPSRFSGSGSGTDFTLTISSLQPEDFATYYCHQR |
| | | SSSPTFGQGTKLEIK |
| 26 | AS20 VH | QVQLQQPGAELVKPGASVKMSCKASGYTFTSYNMHWVKQTPGQGL |
| | | EWIGVIYPGNGDTSYNQKFKGKATLTADKSSSTAYMQLSSLTSED |
| | | SAVYYCARDYYGSSPLGYWGQGTTLTVSS |
| 27 | AS20 VL | QIVLTQSPAVMSASPGEKVTMTCSASSSISYMHWYQQKPGTSPKR |
| | | WIYDTSKLASGVPARFSGSGSGTSYSLTISSMEAEDAATYYCHQR |
| | | SSYPTFGGGTKLEIK |
| 28 | AS20 hIgG1 | QVQLQQPGAELVKPGASVKMSCKASGYTFTSYNMHWVKQTPGQGL |
| | LALA-PG HC | EWIGVIYPGNGDTSYNQKFKGKATLTADKSSSTAYMQLSSLTSED |
| | | SAVYYCARDYYGSSPLGYWGQGTTLTVSSASTKGPSVFPLAPSSK |
| | | STSGGTAALGCLVKDYFPEPVTVSWNSGALTSGVHTFPAVLQSSG |
| | | LYSLSSVVTVPSSSLGTQTYICNVNHKPSNTKVDKKVEPKSCDKT |
| | | HTCPPCPAPEAAGGPSVFLFPPKPKDTLMISRTPEVTCVVVDVSH |
| | | EDPEVKFNWYVDGVEVHNAKTKPREEQYNSTYRVVSVLTVLHQDW |
| | | LNGKEYKCKVSNKALGAPIEKTISKAKGQPREPQVYTLPPSRDEL |
| | | TKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 29 | AS20 hIgG1 | QIVLTQSPAVMSASPGEKVTMTCSASSSISYMHWYQQKPGTSPKR |
| | LALA-PG LC | WIYDTSKLASGVPARFSGSGSGTSYSLTISSMEAEDAATYYCHQR |
| | | SSYPTFGGGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSENRGEC |
| 30 | SL048-11 | QVQLVQSGAEVKKPGASVKVSCKASGYTFTSYNMHWVRQAPGQGL |
| | hIgG4 S228P | EWMGVIYPGDGATSYAQKFKGRVTMTRDTSTSTVYMELSSLRSED |
| | HC | TAVYYCARDYYGSSPLGYWGQGTLVTVSSASTKGPSVFPLAPCSR |
| | | STSESTAALGCLVKDYFPEPVTVSWNSGALTSGVHTFPAVLQSSG |
| | | LYSLSSVVTVPSSSLGTKTYTCNVDHKPSNTKVDKRVESKYGPPC |
| | | PPCPAPEFLGGPSVFLFPPKPKDTLMISRTPEVTCVVVDVSQEDP |
| | | EVQFNWYVDGVEVHNAKTKPREEQFNSTYRVVSVLTVLHQDWING |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 31 | SL048-11 | DIQMTQSPSSLSASVGDRVTITCSASPSISYMNWYQQKPGKAPKL |
| | hIgG4 S228P | LIYATSSLASGVPSRFSGSGSGTDFTLTISSLQPEDFATYYCHQR |
| | LC | SSYPTFGQGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSENRGEC |
| 32 | SL048-46 | QVQLVQSGAEVKKPGASVKVSCKASGYTFTSYNMHWVRQAPGQGL |
| | hIgG4 S228P | EWMGVINPGDGATSYNQKFQGRVTMTRDTSTSTVYMELSSLRSED |
| | HC | TAVYYCARDYYGSSPLGYWGQGTLVTVSSASTKGPSVFPLAPCSR |
| | | STSESTAALGCLVKDYFPEPVTVSWNSGALTSGVHTFPAVLQSSG |
| | | LYSLSSVVTVPSSSLGTKTYTCNVDHKPSNTKVDKRVESKYGPPC |
| | | PPCPAPEFLGGPSVFLFPPKPKDTLMISRTPEVTCVVVDVSQEDP |
| | | EVQFNWYVDGVEVHNAKTKPREEQFNSTYRVVSVLTVLHQDWING |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 33 | SL048-46 | DIQMTQSPSSLSASVGDRVTITCRASSSISYLNWYQQKPGKAPKL |
| | hIgG4 S228P | LIYAASSLASGVPSRFSGSGSGTDFTLTISSLQPEDFATYYCHQR |
| | LC | SSSPTFGQGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSENRGEC |
| 34 | SL048-106 | QVQLVQSGAEVKKPGASVKVSCKASGYTFTSYNMHWVRQAPGQGL |
| | hIgG4 S228P | EWIGVIYPGDGSTSYNQKFQGRVTMTRDTSTSTVYMELSSLRSED |
| | HC | TAVYYCARDYYGSSPLGYWGQGTLVTVSSASTKGPSVFPLAPCSR |
| | | STSESTAALGCLVKDYFPEPVTVSWNSGALTSGVHTFPAVLQSSG |
| | | LYSLSSVVTVPSSSLGTKTYTCNVDHKPSNTKVDKRVESKYGPPC |
| | | PPCPAPEFLGGPSVFLFPPKPKDTLMISRTPEVTCVVVDVSQEDP |
| | | EVQFNWYVDGVEVHNAKTKPREEQFNSTYRVVSVLTVLHQDWLNG |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 35 | SL048-106 | DIQMTQSPSSLSASVGDRVTITCSASSSISYMNWYQQKPGKAPKL |
| | hIgG4 S228P | LIYATSKLPSGVPSRFSGSGSGTDFTLTISSLQPEDFATYYCHQR |
| | LC | SSTPTFGQGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSENRGEC |
| 36 | SOL-116 hIgG4 | QVQLVQSGAEVKKPGASVKVSCKASGYTFTSYNMHWVRQAPGQGL |
| | S228P HC | EWMGVINPGDGATSYAQKFKGRVTMTRDTSTSTVYMELSSLRSED |
| | | TAVYYCARDYYGSSPLGYWGQGTLVTVSSASTKGPSVFPLAPCSR |
| | | STSESTAALGCLVKDYFPEPVTVSWNSGALTSGVHTFPAVLQSSG |
| | | LYSLSSVVTVPSSSLGTKTYTCNVDHKPSNTKVDKRVESKYGPPC |
| | | PPCPAPEFLGGPSVFLFPPKPKDTLMISRTPEVTCVVVDVSQEDP |
| | | EVQFNWYVDGVEVHNAKTKPREEQFNSTYRVVSVLTVLHQDWLNG |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 37 | SOL-116 hIgG4 | DIQMTQSPSSLSASVGDRVTITCRASPSISYMNWYQQKPGKAPKL |
| | S228P LC | LIYATSSLASGVPSRFSGSGSGTDFTLTISSLQPEDFATYYCHQR |
| | | SSSPTFGQGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSENRGEC |
| 38 | SL048-118 | QVQLVQSGAEVKKPGASVKVSCKASGYTFTSYNMHWVRQAPGQGL |
| | hIgG4 S228P | EWMGVINPGDGATSYAQKFKGRVTMTRDTSTSTVYMELSSLRSED |
| | HC | TAVYYCARDYYGSSPLGYWGQGTLVTVSSASTKGPSVFPLAPCSR |
| | | STSESTAALGCLVKDYFPEPVTVSWNSGALTSGVHTFPAVLQSSG |
| | | LYSLSSVVTVPSSSLGTKTYTCNVDHKPSNTKVDKRVESKYGPPC |
| | | PPCPAPEFLGGPSVFLFPPKPKDTLMISRTPEVTCVVVDVSQEDP |
| | | EVQFNWYVDGVEVHNAKTKPREEQFNSTYRVVSVLIVLHQDWING |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 39 | SL048-118 | DIQMTQSPSSLSASVGDRVTITCSASPSISYMNWYQQKPGKAPKL |
| | hIgG4 S228P | LIYATSSLPSGVPSRESGSGSGTDFTLTISSLQPEDFATYYCHQR |
| | LC | SSSPTFGQGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSENRGEC |
| 40 | AS20 hIgG4 | QVQLQQPGAELVKPGASVKMSCKASGYTFTSYNMHWVKQTPGQGL |
| | S228P HC | EWIGVIYPGNGDTSYNQKFKGKATLTADKSSSTAYMQLSSLTSED |
| | | SAVYYCARDYYGSSPLGYWGQGTTLTVSSASTKGPSVFPLAPCSR |
| | | STSESTAALGCLVKDYFPEPVTVSWNSGALTSGVHTFPAVLQSSG |
| | | LYSLSSVVTVPSSSLGTKTYTCNVDHKPSNTKVDKRVESKYGPPC |
| | | PPCPAPEFLGGPSVFLFPPKPKDTLMISRTPEVTCVVVDVSQEDP |
| | | EVQFNWYVDGVEVHNAKTKPREEQFNSTYRVVSVLTVLHQDWLNG |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 41 | AS20 hIgG4 | QIVLTQSPAVMSASPGEKVTMTCSASSSISYMHWYQQKPGTSPKR |
| | S228P LC | WIYDTSKLASGVPARFSGSGSGTSYSLTISSMEAEDAATYYCHQR |
| | | SSYPTFGGGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSENRGEC |
| 42 | CDR graft | QVQLVQSGAEVKKPGASVKVSCKASGYTFTSYNMHWVRQAPGQGL |
| | hIgG4 S228P | EWIGVIYPGNGDTSYNQKFKGRVTMTRDTSTSTVYMELSSLRSED |
| | HC | TAVYYCARDYYGSSPLGYWGQGTLVTVSSASTKGPSVFPLAPCSR |
| | | STSESTAALGCLVKDYFPEPVTVSWNSGALTSGVHTFPAVLQSSG |
| | | LYSLSSVVTVPSSSLGTKTYTCNVDHKPSNTKVDKRVESKYGPPC |
| | | PPCPAPEFLGGPSVFLFPPKPKDTLMISRTPEVTCVVVDVSQEDP |
| | | EVQFNWYVDGVEVHNAKTKPREEQFNSTYRVVSVLTVLHQDWING |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 43 | CDR graft | DIQMTQSPSSLSASVGDRVTITCSASSSISYMHWYQQKPGKAPKL |
| | hIgG4 S228P | LIYDTSKLASGVPSRFSGSGSGTDFTLTISSLQPEDFATYYCHQR |
| | LC | SSYPTFGQGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSENRGEC |
| 44 | AS20 IgG HC | MGWSCIILFLVATATGVHSQVQLQQPGAELVKPGASVKMSCKASG |
| | | YTFTSYNMHWVKQTPGQGLEWIGVIYPGNGDTSYNQKFKGKATLT |
| | | ADKSSSTAYMQLSSLTSEDSAVYYCARDYYGSSPLGYWGQGTTLT |
| | | VSS |
| 45 | AS20 IgG LC | MDFQVQIFSFLLISASVILSRGQIVLTQSPAVMSASPGEKVTMTC |
| | | SASSSISYMHWYQQKPGTSPKRWIYDTSKLASGVPARFSGSGSGT |
| | | SYSLTISSMEAEDAATYYCHQRSSYPTFGGGTKLEIK |
| 46 | hBSSL | AKLGAVYTEG GFVEGVNKKL GLLGDSVDIF KGIPFAAPTK |
| | | ALENPQPHPG WQGTLKAKNF KKRCLQATIT QDSTYGDEDC |
| | | LYLNIWVPQG RKQVSRDLPV MIWIYGGAFL MGSGHGANFL |
| | | NNYLYDGEEI ATRGNVIVVT FNYRVGPLGF LSTGDANLPG |
| | | NYGLRDQHMA IAWVKRNIAA FGGDPNNITL FGESAGGASV |
| | | SLQTLSPYNK GLIRRAISQS GVALSPWVIQ KNPLFWAKKV |
| | | AEKVGCPVGD AARMAQCLKV TDPRALTLAY KVPLAGLEYP |
| | | MLHYVGFVPV IDGDFIPADP INLYANAADI DYIAGTNNMD |
| | | GHIFASIDMP AINKGNKKVT EEDFYKLVSE FTITKGLRGA |
| | | KTTFDVYTES WAQDPSQENK KKTVVDFETD VLFLVPTEIA |
| | | LAQHRANAKS AKTYAYLFSH PSRMPVYPKW VGADHADDIQ |
| | | YVFGKPFATP TGYRPQDRTV SKAMIAYWIN FAKTGDPNMG |
| | | DSAVPTHWEP YTTENSGYLE ITKKMGSSSM KRSLRTNFLR |
| | | YWTLTYLALP TVTDQEATPV PPTGDSEATP VPPTGDSETA |
| | | PVPPTGDSGA PPVPPTGDSG APPVPPTGDS GAPPVPPTGD |
| | | SGAPPVPPTG DSGAPPVPPT GDSGAPPVPP TGDSGAPPVP |
| | | PTGDAGPPPV PPTGDSGAPP VPPTGDSGAP PVTPTGDSET |
| | | APVPPTGDSG APPVPPTGDS EAAPVPPTDD SKEAQMPAVI |
| | | RF |
| 47 | chimeric AS20 | QVQLQQPGAELVKPGASVKMSCKASGYTFTSYNMHWVKQTPGQGL |
| | IgG4 HC | EWIGVIYPGNGDTSYNQKFKGKATLTADKSSSTAYMQLSSLTSED |
| | | SAVYYCARDYYGSSPLGYWGQGTTLTVSSASTKGPSVFPLAPCSR |
| | | STSESTAALGCLVKDYFPEPVTVSWNSGALTSGVHTFPAVLQSSG |
| | | LYSLSSVVTVPSSSLGTKTYTCNVDHKPSNTKVDKRVESKYGPPC |
| | | PSCPAPEFLGGPSVFLFPPKPKDTLMISRTPEVTCVVVDVSQEDP |
| | | EVQFNWYVDGVEVHNAKTKPREEQFNSTYRVVSVLTVLHQDWING |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 48 | chimeric AS20 | QIVLTQSPAVMSASPGEKVTMTCSASSSISYMHWYQQKPGTSPKR |
| | IgG4 LC | WIYDTSKLASGVPARFSGSGSGTSYSLTISSMEAEDAATYYCHQR |
| | | SSYPTFGGGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSENRGEC |
| 49 | HCDR1 (AS20) | GYFTSYN |
| 50 | HCDR2 (AS20) | IGVIYPGNGDTSYNQKFK |
| 51 | HCDR3 (AS20) | ARDYYGSSPLGY |
| 52 | LCDR1 (AS20) | SASSSISYMH |
| 53 | LCDR2 (AS20) | DTSKLA |

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible.

1.-25. (canceled)

26. A method for treating a bile salt-stimulated lipase (BSSL) expressing cancer in a subject, the method comprises administering a therapeutically effective amount of an antibody, or an antigen-binding fragment thereof, or a pharmaceutical composition comprising the antibody, or the antigen-binding fragment thereof, to a subject in need thereof, wherein the antibody, or the antigen-binding fragment thereof, binds to an epitope present in an N-terminal part of BSSL ranging from a N-terminus up to amino acid residue 300 in BSSL but excludes an active site of BSSL.

27. The method according to claim 26, wherein the antibody, or the antigen-binding fragment thereof, does not affect an enzymatic lipase activity of BBSL.

28. The method according to claim 26, wherein the antibody, or the antigen-binding fragment thereof, binds to an epitope of BSSL comprising:
a first surface comprising an amino acid sequence according to SEQ ID NO: 1; and
a second surface comprising an amino acid sequence according to SEQ ID NO: 2.

29. The method according to claim 28, wherein the first surface comprises an amino acid sequence according to SEQ ID NO: 3.

30. The method according to claim 28, wherein the antibody, or the antigen-binding fragment thereof, further specifically binds to a surface selected from the group consisting of:
an amino acid sequence according to SEQ ID NO: 5;
an amino acid sequence according to SEQ ID NO: 4; and
an amino acid sequence according to SEQ ID NO: 6.

31. The method according to claim 28, wherein the antibody, or antigen-binding fragment thereof, comprises:
three complementarity determining regions (CDRs) of a heavy chain variable region (HCVR) (HCDRs); and
three CDRs of a light chain variable region (LCVR) (LCDRs), wherein
a first HCDR comprises an amino acid sequence according to SEQ ID NO: 7;
a second HCDR comprises an amino acid sequence selected from the group consisting of SEQ ID NO: 8, 12 and 13;
a third HCDR comprises an amino acid sequence according to SEQ ID NO: 9;
a first LCDR comprises an amino acid sequence selected from the group consisting of SEQ ID NO: 10 and 14; and
a third LCDR comprises an amino acid sequence selected from the group consisting of SEQ ID NO: 11, 15 and 16.

32. The method according to claim 31, wherein the antibody, or antigen-binding fragment thereof, comprises a second LCDR comprises an amino acid sequence selected from the group consisting of ATS and AAS.

33. The method according to claim 31, wherein
the first HCDR consists of an amino acid sequence according to SEQ ID NO: 7;
the second HCDR consists of an amino acid sequence selected from the group consisting of SEQ ID NO: 8, 12 and 13;
the third HCDR consists of an amino acid sequence according to SEQ ID NO: 9;
the first LCDR consists of an amino acid sequence selected from the group consisting of SEQ ID NO: 10 and 14; and
the third LCDR consists of an amino acid sequence selected from the group consisting of SEQ ID NO: 11, 15 and 16.

34. The method according to claim 33, wherein the antibody, or antigen-binding fragment thereof, comprises a second LCDR consists of an amino acid sequence selected from the group consisting of ATS and AAS.

35. The method according to claim 31, wherein
the first HCDR comprises the amino acid sequence according to SEQ ID NO: 7;
the second HCDR comprises the amino acid sequence according to SEQ ID NO: 8;
the third HCDR comprises the amino acid sequence according to SEQ ID NO: 9;
the first LCDR comprises the amino acid sequence according to SEQ ID NO: 10;
the second LCDR comprises the amino acid sequence ATS; and
the third LCDR comprises the amino acid sequence according to SEQ ID NO: 11.

36. The method according to claim 35, wherein
the HCVR comprises an amino acid sequence according to SEQ ID NO: 23; and/or
the LCVR comprises an amino acid sequence selected from the group consisting of SEQ ID NO: 24 and 25.

37. The method according to claim 36, wherein
a heavy chain (HC) comprises an amino acid sequence according to SEQ ID NO: 36; and/or
a light chain (LC) comprises an amino acid sequence according to SEQ ID NO: 37.

38. The method according to claim 36, wherein
a heavy chain (HC) comprises an amino acid sequence according to SEQ ID NO: 38; and/or
a light chain (LC) comprises an amino acid sequence according to SEQ ID NO: 39.

39. The method according to claim 31, wherein
the first HCDR comprises the amino acid sequence according to SEQ ID NO: 7;
the second HCDR comprises the amino acid sequence according to SEQ ID NO: 12;
the third HCDR comprises the amino acid sequence according to SEQ ID NO: 9;
the first LCDR comprises the amino acid sequence according to SEQ ID NO: 10;
the second LCDR comprises the amino acid sequence ATS; and
the third LCDR comprises the amino acid sequence according to SEQ ID NO: 15.

40. The method according to claim 38, wherein
the HCVR comprises an amino acid sequence according to SEQ ID NO: 17; and/or
the LCVR comprises an amino acid sequence according to SEQ ID NO: 18.

41. The method according to claim 40, wherein
a heavy chain (HC) comprises an amino acid sequence according to SEQ ID NO: 30; and/or
a light chain (LC) comprises an amino acid sequence according to SEQ ID NO: 31.

42. The method according to claim 31, wherein
the first HCDR comprises the amino acid sequence according to SEQ ID NO: 7;
the second HCDR comprises the amino acid sequence according to SEQ ID NO: 8;
the third HCDR comprises the amino acid sequence according to SEQ ID NO: 9;
the first LCDR comprises the amino acid sequence according to SEQ ID NO: 14;
the second LCDR comprises the amino acid sequence AAS; and
the third LCDR comprises the amino acid sequence according to SEQ ID NO: 11.

43. The method according to claim 42, wherein
the HCVR comprises an amino acid sequence according to SEQ ID NO: 19; and/or
the LCVR comprises an amino acid sequence according to SEQ ID NO: 20.

44. The method according to claim 43, wherein
a heavy chain (HC) comprises an amino acid sequence according to SEQ ID NO: 32; and/or
a light chain (LC) comprises an amino acid sequence according to SEQ ID NO: 33.

45. The method according to claim 31, wherein
the first HCDR comprises the amino acid sequence according to SEQ ID NO: 7;
the second HCDR comprises the amino acid sequence according to SEQ ID NO: 13;
the third HCDR comprises the amino acid sequence according to SEQ ID NO: 9;
the first LCDR comprises the amino acid sequence according to SEQ ID NO: 14;
the second LCDR comprises the amino acid sequence ATS; and
the third LCDR comprises the amino acid sequence according to SEQ ID NO: 16.

46. The method according to claim 45, wherein
the HCVR comprises an amino acid sequence according to SEQ ID NO: 21; and/or
the LCVR comprises an amino acid sequence according to SEQ ID NO: 22.

47. The method according to claim 46, wherein
a heavy chain (HC) comprises an amino acid sequence according to SEQ ID NO: 34; and/or
a light chain (LC) comprises an amino acid sequence according to SEQ ID NO: 35.

48. The method according to claim 26, wherein the antibody, or antigen-binding fragment thereof, comprises:
three complementarity determining regions (CDRs) of a heavy chain variable region (HCVR) (HCDRs); and
three CDRs of a light chain variable region (LCVR) (LCDRs), wherein
a first HCDR comprises the amino acid sequence according to SEQ ID NO: 49;
a second HCDR comprises the amino acid sequence according to SEQ ID NO: 50
a third HCDR comprises the amino acid sequence according to SEQ ID NO: 51;
a first LCDR comprises the amino acid sequence according to SEQ ID NO: 52
a second LCDR comprises the amino acid sequence according to SEQ ID NO: 53; and
a third LCDR comprises, preferably consists of, the amino acid sequence according to SEQ ID NO: 15.

49. The method according to claim 48, wherein
a heavy chain (HC) comprises an amino acid sequence selected from the group consisting of SEQ ID NO: 28, 40, 42, and 47; and/or
a light chain (LC) comprises an amino acid sequence selected from the group consisting of SEQ ID NO: 29, 41, 43, and 48.

50. The method according to claim 26, wherein the BSSL-expressing cancer is selected from the group consisting of colon cancer, lung cancer, colorectal cancer, pancreatic cancer, breast cancer, prostate cancer and liver cancer.

51. The method according to claim 26, wherein the BSSL-expressing cancer is characterized by carboxyl ester lipase (CEL) messenger ribonucleic acid (mRNA) transcripts per million of at least 25.

52. The method according to claim 51, wherein the BSSL-expressing cancer is characterized by CEK mRNA transcripts per million of at least 50.

53. The method according to claim 26, wherein the BSSL-expressing cancer is characterized by higher BSSL expression than BSSL expression from corresponding healthy tissue, from which the BSSL-expressing cancer is derived.


SEQUENCE LISTING (53 sequences)

SEQ ID NO: 1 - protein, 6 aa, Homo sapiens
YTEGGF

SEQ ID NO: 2 - protein, 14 aa, Homo sapiens
LENPQPHPGWQGTL

SEQ ID NO: 3 - protein, 12 aa, Homo sapiens
AKLGAVYTEGGF

SEQ ID NO: 4 - protein, 18 aa, Homo sapiens
NIWVPQGRKQVSRDLPVM

SEQ ID NO: 5 - protein, 7 aa, Homo sapiens
VKRNIAA

SEQ ID NO: 6 - protein, 13 aa, Homo sapiens
HYVGFVPVIDGDF

SEQ ID NO: 7 - protein, 8 aa, synthetic construct
GYTFTSYN

SEQ ID NO: 8 - protein, 8 aa, synthetic construct
INPGDGAT

SEQ ID NO: 9 - protein, 12 aa, synthetic construct
ARDYYGSSPLGY

SEQ ID NO: 10 - protein, 5 aa, synthetic construct
PSISY

SEQ ID NO: 11 - protein, 8 aa, synthetic construct
HQRSSSPT

SEQ ID NO: 12 - protein, 8 aa, synthetic construct
IYPGDGAT

SEQ ID NO: 13 - protein, 8 aa, synthetic construct
IYPGDGST

SEQ ID NO: 14 - protein, 5 aa, synthetic construct
SSISY

SEQ ID NO: 15 - protein, 8 aa, synthetic construct
HQRSSYPT

SEQ ID NO: 16 - protein, 8 aa, synthetic construct
HQRSSTPT

SEQ ID NO: 17 - protein, 119 aa, synthetic construct
QVQLVQSGAEVKKPGASVKVSCKASGYTFTSYNMHWVRQAPGQGLEWMGVIYPGDGATSYAQKFKGRVTMTRDTSTSTVYMELSSLRSEDTAVYYCARDYYGSSPLGYWGQGTLVTVSS

SEQ ID NO: 18 - protein, 105 aa, synthetic construct
DIQMTQSPSSLSASVGDRVTITCSASPSISYMNWYQQKPGKAPKLLIYATSSLASGVPSRFSGSGSGTDFTLTISSLQPEDFATYYCHQRSSYPTFGQGTKLEIK

SEQ ID NO: 19 - protein, 119 aa, synthetic construct
QVQLVQSGAEVKKPGASVKVSCKASGYTFTSYNMHWVRQAPGQGLEWMGVINPGDGATSYNQKFQGRVTMTRDTSTSTVYMELSSLRSEDTAVYYCARDYYGSSPLGYWGQGTLVTVSS

SEQ ID NO: 20 - protein, 105 aa, synthetic construct
DIQMTQSPSSLSASVGDRVTITCRASSSISYLNWYQQKPGKAPKLLIYAASSLASGVPSRFSGSGSGTDFTLTISSLQPEDFATYYCHQRSSSPTFGQGTKLEIK

SEQ ID NO: 21 - protein, 119 aa, synthetic construct
QVQLVQSGAEVKKPGASVKVSCKASGYTFTSYNMHWVRQAPGQGLEWIGVIYPGDGSTSYNQKFQGRVTMTRDTSTSTVYMELSSLRSEDTAVYYCARDYYGSSPLGYWGQGTLVTVSS

SEQ ID NO: 22 - protein, 105 aa, synthetic construct
DIQMTQSPSSLSASVGDRVTITCSASSSISYMNWYQQKPGKAPKLLIYATSKLPSGVPSRFSGSGSGTDFTLTISSLQPEDFATYYCHQRSSTPTFGQGTKLEIK

SEQ ID NO: 23 - protein, 119 aa, synthetic construct
QVQLVQSGAEVKKPGASVKVSCKASGYTFTSYNMHWVRQAPGQGLEWMGVINPGDGATSYAQKFKGRVTMTRDTSTSTVYMELSSLRSEDTAVYYCARDYYGSSPLGYWGQGTLVTVSS

SEQ ID NO: 24 - protein, 105 aa, synthetic construct
DIQMTQSPSSLSASVGDRVTITCRASPSISYMNWYQQKPGKAPKLLIYATSSLASGVPSRFSGSGSGTDFTLTISSLQPEDFATYYCHQRSSSPTFGQGTKLEIK

SEQ ID NO: 25 - protein, 105 aa, synthetic construct
DIQMTQSPSSLSASVGDRVTITCSASPSISYMNWYQQKPGKAPKLLIYATSSLPSGVPSRFSGSGSGTDFTLTISSLQPEDFATYYCHQRSSSPTFGQGTKLEIK

SEQ ID NO: 26 - protein, 119 aa, synthetic construct
QVQLQQPGAELVKPGASVKMSCKASGYTFTSYNMHWVKQTPGQGLEWIGVIYPGNGDTSYNQKFKGKATLTADKSSSTAYMQLSSLTSEDSAVYYCARDYYGSSPLGYWGQGTTLTVSS

SEQ ID NO: 27 - protein, 105 aa, synthetic construct
QIVLTQSPAVMSASPGEKVTMTCSASSSISYMHWYQQKPGTSPKRWIYDTSKLASGVPARFSGSGSGTSYSLTISSMEAEDAATYYCHQRSSYPTFGGGTKLEIK

SEQ ID NO: 28 - protein, 449 aa, synthetic construct
QVQLQQPGAELVKPGASVKMSCKASGYTFTSYNMHWVKQTPGQGLEWIGVIYPGNGDTSYNQKFKGKATLTADKSSSTAYMQLSSLTSEDSAVYYCARDYYGSSPLGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 212 aa, synthetic construct
QIVLTQSPAVMSASPGEKVTMTCSASSSISYMHWYQQKPGTSPKRWIYDTSKLASGVPARFSGSGSGTSYSLTISSMEAEDAATYYCHQRSSYPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 446 aa, synthetic construct
QVQLVQSGAEVKKPGASVKVSCKASGYTFTSYNMHWVRQAPGQGLEWMGVIYPGDGATSYAQKFKGRVTMTRDTSTSTVYMELSSLRSEDTAVYYCARDYYGSSPLGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 212 aa, synthetic construct
DIQMTQSPSSLSASVGDRVTITCSASPSISYMNWYQQKPGKAPKLLIYATSSLASGVPSRFSGSGSGTDFTLTISSLQPEDFATYYCHQRSSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 446 aa, synthetic construct
QVQLVQSGAEVKKPGASVKVSCKASGYTFTSYNMHWVRQAPGQGLEWMGVINPGDGATSYNQKFQGRVTMTRDTSTSTVYMELSSLRSEDTAVYYCARDYYGSSPLGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 212 aa, synthetic construct
DIQMTQSPSSLSASVGDRVTITCRASSSISYLNWYQQKPGKAPKLLIYAASSLASGVPSRFSGSGSGTDFTLTISSLQPEDFATYYCHQRSSSPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 446 aa, synthetic construct
QVQLVQSGAEVKKPGASVKVSCKASGYTFTSYNMHWVRQAPGQGLEWIGVIYPGDGSTSYNQKFQGRVTMTRDTSTSTVYMELSSLRSEDTAVYYCARDYYGSSPLGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 212 aa, synthetic construct
DIQMTQSPSSLSASVGDRVTITCSASSSISYMNWYQQKPGKAPKLLIYATSKLPSGVPSRFSGSGSGTDFTLTISSLQPEDFATYYCHQRSSTPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 446 aa, synthetic construct
QVQLVQSGAEVKKPGASVKVSCKASGYTFTSYNMHWVRQAPGQGLEWMGVINPGDGATSYAQKFKGRVTMTRDTSTSTVYMELSSLRSEDTAVYYCARDYYGSSPLGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 212 aa, synthetic construct
DIQMTQSPSSLSASVGDRVTITCRASPSISYMNWYQQKPGKAPKLLIYATSSLASGVPSRFSGSGSGTDFTLTISSLQPEDFATYYCHQRSSSPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 446 aa, synthetic construct; identical to SEQ ID NO: 36

SEQ ID NO: 39 - protein, 212 aa, synthetic construct
DIQMTQSPSSLSASVGDRVTITCSASPSISYMNWYQQKPGKAPKLLIYATSSLPSGVPSRFSGSGSGTDFTLTISSLQPEDFATYYCHQRSSSPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 446 aa, synthetic construct
QVQLQQPGAELVKPGASVKMSCKASGYTFTSYNMHWVKQTPGQGLEWIGVIYPGNGDTSYNQKFKGKATLTADKSSSTAYMQLSSLTSEDSAVYYCARDYYGSSPLGYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 212 aa, synthetic construct; identical to SEQ ID NO: 29

SEQ ID NO: 42 - protein, 446 aa, synthetic construct
QVQLVQSGAEVKKPGASVKVSCKASGYTFTSYNMHWVRQAPGQGLEWIGVIYPGNGDTSYNQKFKGRVTMTRDTSTSTVYMELSSLRSEDTAVYYCARDYYGSSPLGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - protein, 212 aa, synthetic construct
DIQMTQSPSSLSASVGDRVTITCSASSSISYMHWYQQKPGKAPKLLIYDTSKLASGVPSRFSGSGSGTDFTLTISSLQPEDFATYYCHQRSSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 138 aa, synthetic construct
MGWSCIILFLVATATGVHSQVQLQQPGAELVKPGASVKMSCKASGYTFTSYNMHWVKQTPGQGLEWIGVIYPGNGDTSYNQKFKGKATLTADKSSSTAYMQLSSLTSEDSAVYYCARDYYGSSPLGYWGQGTTLTVSS

SEQ ID NO: 45 - protein, 127 aa, synthetic construct
MDFQVQIFSFLLISASVILSRGQIVLTQSPAVMSASPGEKVTMTCSASSSISYMHWYQQKPGTSPKRWIYDTSKLASGVPARFSGSGSGTSYSLTISSMEAEDAATYYCHQRSSYPTFGGGTKLEIK

SEQ ID NO: 46 - protein, 722 aa, Homo sapiens
AKLGAVYTEGGFVEGVNKKLGLLGDSVDIFKGIPFAAPTKALENPQPHPGWQGTLKAKNFKKRCLQATITQDSTYGDEDCLYLNIWVPQGRKQVSRDLPVMIWIYGGAFLMGSGHGANFLNNYLYDGEEIATRGNVIVVTFNYRVGPLGFLSTGDANLPGNYGLRDQHMAIAWVKRNIAAFGGDPNNITLFGESAGGASVSLQTLSPYNKGLIRRAISQSGVALSPWVIQKNPLFWAKKVAEKVGCPVGDAARMAQCLKVTDPRALTLAYKVPLAGLEYPMLHYVGFVPVIDGDFIPADPINLYANAADIDYIAGTNNMDGHIFASIDMPAINKGNKKVTEEDFYKLVSEFTITKGLRGAKTTFDVYTESWAQDPSQENKKKTVVDFETDVLFLVPTEIALAQHRANAKSAKTYAYLFSHPSRMPVYPKWVGADHADDIQYVFGKPFATPTGYRPQDRTVSKAMIAYWTNFAKTGDPNMGDSAVPTHWEPYTTENSGYLEITKKMGSSSMKRSLRTNFLRYWTLTYLALPTVTDQEATPVPPTGDSEATPVPPTGDSETAPVPPTGDSGAPPVPPTGDSGAPPVPPTGDSGAPPVPPTGDSGAPPVPPTGDSGAPPVPPTGDSGAPPVPPTGDSGAPPVPPTGDAGPPPVPPTGDSGAPPVPPTGDSGAPPVTPTGDSETAPVPPTGDSGAPPVPPTGDSEAAPVPPTDDSKEAQMPAVIRF

SEQ ID NO: 47 - protein, 446 aa, synthetic construct
QVQLQQPGAELVKPGASVKMSCKASGYTFTSYNMHWVKQTPGQGLEWIGVIYPGNGDTSYNQKFKGKATLTADKSSSTAYMQLSSLTSEDSAVYYCARDYYGSSPLGYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 212 aa, synthetic construct; identical to SEQ ID NO: 29

SEQ ID NO: 49 - protein, 7 aa, synthetic construct
GYFTSYN

SEQ ID NO: 50 - protein, 18 aa, synthetic construct
IGVIYPGNGDTSYNQKFK

SEQ ID NO: 51 - protein, 12 aa, synthetic construct
ARDYYGSSPLGY

SEQ ID NO: 52 - protein, 10 aa, synthetic construct
SASSSISYMH

SEQ ID NO: 53 - protein, 6 aa, synthetic construct
DTSKLA